(19) United States
(12) Patent Application Publication
(10) Pub. No.: US 2025/0205152 A1
(43) Pub. Date: Jun. 26, 2025

(54) METHOD OF PREVENTING AGE-RELATED MACULAR DEGENERATION BY ADMINISTERING AN OCULAR DRUG DELIVERY INSERT

(71) Applicant: EYEPOINT PHARMACEUTICALS, INC., Watertown, MA (US)

(72) Inventors: Said SAIM, Watertown, MA (US); Jay S. DUKER, Watertown, MA (US); Dario PAGGIARINO, Watertown, MA (US); Michelle HOWARD-SPARKS, Watertown, MA (US)

(21) Appl. No.: 18/843,566

(22) PCT Filed: Mar. 10, 2023
(86) PCT No.: PCT/US2023/064139

§ 371 Date: Sep. 3, 2024

Related U.S. Application Data
(60) Provisional application No. 63/390,036, filed on Jul. 18, 2022.
(60) Provisional application No. 63/318,822, filed on Mar. 11, 2022.

(51) Int. Cl. A61K 9/00; A61K 31/404; A61K 47/32

(52) U.S. Cl. CPC A61K 9/0051; A61K 31/404; A61K 47/32

(57) ABSTRACT

This invention relates to a method for preventing, stabilizing or slowing progression of AMD in an eye in a human subject comprising administering to the eye an ocular drug delivery insert.

### BACKGROUND OF THE INVENTION

### Background

Age-related macular degeneration (“AMD”) is a leading cause of blindness worldwide. AMD causes the progressive loss of central vision attributable to degenerative and/or neovascular changes in the macula, a specialized area in the center of the retina. In general, macular degeneration can produce a slow or sudden loss of vision. AMD is estimated to affect almost 200 million people around the world, with late-stage AMD affecting almost 11 million people.

Two forms of AMD exist: dry AMD and wet AMD. Typically, AMD begins as dry AMD, which is characterized by the formation of drusen, yellow plaque-like deposits in the macula between the retinal pigment epithelium and the underlying choroid. Dry AMD may progress to wet AMD.

Dry macular degeneration is more common than wet AMD, with about 90% of AMD patients being diagnosed with dry AMD. The dry form of AMD may result from the aging and thinning of macular tissues, depositing of pigment in the macula, or a combination of the two processes.

The wet form of the disease usually leads to more serious vision loss. Wet AMD is characterized by the formation of new blood vessels in the choroid (choroidal neovascularization), macular atrophy (geographic atrophy) and vision loss. With wet AMD, the new blood vessels grow beneath the retina and leak blood and fluid. This leakage causes retinal cells to die and creates blind spots in central vision.

A person may have AMD in one eye, or may have it in both eyes, but may be at different stages of AMD in each eye. Wet AMD typically occurs first in one eye, referred to as unilateral wet AMD. Patients having wet AMD in one eye have a significant risk of developing choroidal neovascularization in their fellow eye. A retrospective study that included patients with unilateral wet AMD treated with anti-vascular endothelial growth factor (VEGF) therapy with at least three years of follow-up after the start of treatment, found that in 38% of these patients, fellow eye conversion took place within a three-year window after onset of wet AMD in the first eye.

There are some treatments for wet AMD, but existing treatments are inconvenient or have significant adverse effects, and they do not cure wet AMD. Moreover, there is currently no drug to prevent wet AMD.

### BRIEF SUMMARY OF THE INVENTION

In accordance with various embodiments of the invention and after extensive experimentation, the inventors have invented a novel bioerodible drug delivery insert comprising an active pharmaceutical ingredient (API) and a bioerodible polymer, and methods of using this insert. This insert is particularly useful for local delivery of an effective amount of the API to the eye. In addition, the insert provides sustained release of the API. In some aspects the insert provides sustained release of API for a period that is nearly synchronized with the period required for complete erosion of the insert in an eye.

These inserts may be administered intraocularly, e.g., intravitreally, suprachoroidally, intracamerally, or subconjunctivally. For example, the inserts may be placed through a needle or cannula for an intravitreal injection. Thus, in some aspects, the invention relates to a drug delivery insert that can deliver effective intraocular concentrations of the API while delivering low systemic concentrations of the API to reduce the risk of toxicity or other undesirable side effects.

Thus, in some aspects, the invention relates to use of the insert for treating or preventing ocular diseases described herein by local (e.g., intraocular) administration of an API or a pharmaceutically acceptable salt thereof.

In some embodiments, the invention provides a method for preventing wet AMD in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days.

In other aspects, the invention provides a method of preventing choroidal neovascularization in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days.

In yet other embodiments, the invention provides a method of preventing conversion of category 3 AMD to category 4 AMD in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days, wherein the eye has category 3 AMD at baseline.

In other aspects, the invention provides a method of slowing the progression of AMD in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days, wherein the eye has category 2, category 3 or category 4 AMD at baseline.

In some aspects, the method for preventing wet AMD in an eye in a human subject comprises administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days.

In other aspects, the method of preventing choroidal neovascularization in an eye in a human subject comprises administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days.

In other embodiments, the method of preventing conversion of category 3 AMD to category 4 AMD in an eye in a human subject comprises administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days, wherein the eye has category 3 AMD at baseline.

In some embodiments, the method of slowing the progression of AMD in an eye in a human subject comprises administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days, wherein the eye has category 2, category 3 or category 4 AMD at baseline.

In certain aspects of these methods the subject has unilateral wet AMD in the subject's other eye at baseline.

In other aspects of the method, at baseline, the eye is in category 1 or has category 2 AMD. In some embodiments, the subject has category 3 AMD in the subject's other eye at baseline. In other embodiments, the subject has category 3 AMD in both eyes at baseline. In yet other embodiments, at baseline, the eye has category 3 AMD and the subject's other eye has category 4 AMD. In some aspects of the method at baseline, the eye has category 2 AMD and the subject's other eye has category 4 AMD. In other aspects, at baseline, the eye is category 1 and the subject's other eye has category 4 AMD.

In some embodiments of the method, an ocular drug delivery insert comprising vorolanib, or a pharmaceutically acceptable salt thereof, that releases about 0.1 μg/day to about 40 μg/day of vorolanib for at least 90 days and is capable of at least 20% erosion within 95 days, is administered to both of the subject's eyes.

In addition, the invention includes a method of providing neuroprotection to an ocular tissue in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days.

The invention also provides a method of preventing the loss of visual acuity due to damage to or loss of retinal cells, such as retinal neurons, in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days. The invention further provides a method of reducing the occurrence of loss of vision due to damage to or loss of retinal cells, such as retinal neurons, in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days. In some embodiments of these methods, the administration of the ocular drug delivery insert reduces retinal thinning in the eye. In other embodiments, the administration of the ocular drug delivery insert protects against photoreceptor degeneration in the eye. The retinal cells may be photoreceptors, bipolar cells, ganglion cells, horizontal cells, or amacrine cells.

In some embodiments, the invention provides a method for treatment or prophylaxis of an ocular disease, comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days, wherein the ocular disease is characterized by damage to retinal neurons. In some aspects of this method, the damage to retinal neurons affects photoreceptors. In other aspects of this method, the ocular disease is geographic atrophy, wAMD, glaucoma, diabetic macular edema, or retinal detachment.

In some embodiments of the methods, there is no change from baseline in BCVA of the eye for 180 days starting on the day on which the insert is administered. In other embodiments, for 180 days starting on the day on which the insert is administered, the change from baseline in BCVA of the eye is a loss of ≤5 ETDRS letters. In yet other embodiments, for 180 days starting on the day on which the insert is administered, CST of the eye does not increase by more than 75 μm.

In other embodiments, CST of the eye does not increase over baseline.

In certain aspects of the method, there is no detectable choroidal neovascularization in the eye for 180 days starting on the day on which the insert is administered.

In other aspects, for at least 180 days starting on the day on which the insert is administered, the eye does not progress to an AMD category higher than the eye was at baseline.

In some aspects of the method, for at least 180 days, starting on the day on which the insert is administered, the IVI questionnaire composite score for the subject does not increase significantly from baseline.

Additional embodiments of the inserts that may be used in the methods of the invention are described herein. Thus, the methods described above are not limited to administration of inserts having only the characteristics described above, such as drug release rate and insert erosion rate.

For example, in some embodiments, the insert comprises a solid matrix core comprising vorolanib, or a pharmaceutically acceptable salt thereof, and a matrix polymer. In some aspects, the matrix polymer is polyvinyl alcohol (PVA). In some embodiments, the amount of matrix polymer in the insert is about 1% w/w to about 15% w/w.

In some embodiments, the amount of vorolanib, or pharmaceutically acceptable salt thereof, in the insert is about 60% w/w to about 98% w/w. In other embodiments, the amount of vorolanib, or pharmaceutically acceptable salt thereof, in the insert is about 85% w/w to about 99% w/w.

In yet other embodiments, the insert is capable of at least 90% erosion within 440 days.

In some embodiments, the insert comprises about 200 μg to about 2000 μg of vorolanib or a pharmaceutically acceptable salt thereof.

In yet other embodiments, the insert releases about 0.5 μg/day to about 30 μg/day of vorolanib for at least 120 days.

In some aspects of the method, the insert is administered by intravitreal injection through a 20 to 27 gauge needle or cannula.

In some aspects, the insert has a length of about 1 mm to about 10 mm.

In some embodiments, the insert further comprises a coating substantially surrounding the core. In yet other embodiments, the coating comprises PVA. In other embodiments, the insert further comprises a delivery port.

In some aspects, the insert matrix polymer is PVA, and the coating comprises a different grade of PVA than the matrix polymer.

In yet other embodiments, the coating comprises at least two coats comprising PVA, and wherein at least one of the coats comprises a different grade of PVA from at least one other coat.

In some embodiments, the coating comprises more than one coat comprising PVA, the matrix polymer is PVA, and the DH of the PVA in at least one coat differs from the DH of the matrix polymer PVA.

In yet other embodiments, the matrix polymer is PVA and the MW of the PVA in the coating differs from the MW of the matrix polymer.

In some aspects of the method, 1-6 inserts are injected. In certain aspects, the total amount of vorolanib in all of the inserts is about 600 μg to about 6000 μg.

In some embodiments, the one or more ocular drug delivery inserts deliver a total average daily dose of vorolanib of about 1 μg/day to about 50 μg/day for at least 30 days.

In some aspects of the method, the insert was cured for about 200 minutes to about 1440 minutes at about 60° C. to about 120° C. In yet other aspects, the insert is made by dissolving PVA in an aqueous solution to form a PVA solution, mixing the PVA solution with vorolanib or a pharmaceutically acceptable salt thereof to form a matrix mixture, extruding the mixture through a dispensing tip to form an elongated shaped matrix, curing the elongated shaped matrix at a temperature of about 80° C. to about 160° C. for about 15 minutes to about 4 hours, and segmenting the elongated shaped matrix.

In some embodiments of the method, the ocular drug delivery insert comprises a solid matrix core comprising a matrix polymer and vorolanib or a pharmaceutically acceptable salt thereof, wherein the amount of the vorolanib or pharmaceutically acceptable salt thereof in the insert is about 10% w/w to about 98% w/w, wherein the drug release rate for the insert is about 0.01 μg/day to about 100 μg/day for at least 14 days and wherein the insert is capable of at least 20% erosion within 95 days. In another embodiment, the amount of the vorolanib or pharmaceutically acceptable salt thereof in the insert is about 60% w/w to about 98% w/w.

In other embodiments, the insert further comprises a coating substantially surrounding the core. In some embodiments, the amount of coating is about 5% w/w to about 20% w/w of the insert. In additional embodiments, the insert further comprises a delivery port.

In some embodiments of the method, the ocular drug delivery insert consists of a solid matrix core comprising an API and at least two different grades of PVA, wherein the drug release rate for the insert is about 0.0001 μg/day to about 200 μg/day for at least 30 days, wherein the insert is capable of at least 20% erosion within 95 days, and wherein the insert is sized and shaped to fit through a 20 to 27 gauge needle or cannula. In some embodiments the two different grades of PVA is a mixture selected from the list comprising: a mixture of MW 78,000, 88% hydrolyzed and MW 78,000, 98% hydrolyzed; a mixture of MW 78,000, 88% hydrolyzed and MW 78,000, 99+% hydrolyzed; a mixture of MW 6,000, 80% hydrolyzed and MW 78,000, 98% hydrolyzed; a mixture of MW 6,000, 80% hydrolyzed and MW 78,000, 99+% hydrolyzed; a mixture of MW 78,000, 88% hydrolyzed and MW 125,000, 88% hydrolyzed; and a mixture of MW 6,000, 80% hydrolyzed and MW 125,000, 88% hydrolyzed.

In other embodiments of the method, the ocular drug delivery insert comprises (a) a solid matrix core comprising PVA and an API, and (b) a coating comprising PVA substantially surrounding the core; wherein the insert comprises at least two different grades of PVA, wherein the insert is capable of at least 20% erosion within 95 days, and wherein the insert is sized and shaped to fit through a 20 to 27 gauge needle or cannula.

In some embodiments of the method, the ocular drug delivery insert comprises:
(a) a solid matrix core comprising a PVA selected from the group consisting of MW 6,000, 80% hydrolyzed, MW 9,000-10,000, 80% hydrolyzed, MW 25,000, 88% hydrolyzed, MW 25,000, 98% hydrolyzed, MW 30,000-70,000, 87-90% hydrolyzed, MW 78,000, 88% hydrolyzed, MW 78,000, 98% hydrolyzed, MW 78,000, 99+% hydrolyzed, MW 89,000-98,000, 99+% hydrolyzed, MW 85,000-124,000, 87-89% hydrolyzed, MW 108,000, 99⁺% hydrolyzed, MW 125,000, 88% hydrolyzed, MW 133,000, 99% hydrolyzed, MW 146,000-186,000, 99+% hydrolyzed, and mixtures thereof; and an API; and
(b) at least one coating comprising PVA substantially surrounding the core, wherein the PVA in the coating is selected from a PVA selected from the group consisting of MW 6,000, 80% hydrolyzed, MW 9,000-10,000, 80% hydrolyzed, MW 25,000, 88% hydrolyzed, MW 25,000, 98% hydrolyzed, MW 30,000-70,000, 87-90% hydrolyzed, MW 78,000, 88% hydrolyzed, MW 78,000, 98% hydrolyzed, MW 78,000, 99+% hydrolyzed, MW 89,000-98,000, 99+% hydrolyzed, MW 85,000-124,000, 87-89% hydrolyzed, MW 108,000, 99+% hydrolyzed, MW 125,000, 88% hydrolyzed, MW 133,000, 99% hydrolyzed, MW 146,000-186,000, 99+% hydrolyzed, and mixtures thereof;
   wherein the PVA in the core and the PVA in at least one coating are different grades of PVA.

In some embodiments of the method, the coating comprises a different grade of PVA than the core PVA. In some embodiments, the DH of the PVA in the coating differs from the DH of the core PVA. In other embodiments, the MW of the PVA in the coating differs from the MW of the core PVA. In some embodiments, the coating comprises at least two coats comprising PVA, and wherein at least one of the coats comprises a different grade of PVA from at least one other coat. In some embodiments, the PVA in at least two coats differ in DH. In some embodiments, the PVA in at least two coats differ in MW.

Additional embodiments of the inserts that may be used in the methods of the invention are described below.

### BRIEF DESCRIPTION OF THE DRAWINGS

FIG. 1 depicts an exemplary ocular drug delivery insert for use in the invention.

FIG. 2 depicts graphs showing the average weight change of films of different grades of PVA after 24 h immersion in PBS.

FIG. 3 depicts a scale showing the relative film strengths of the films evaluated.

FIG. 4A depicts the in vitro drug release profile showing cumulative percent drug release from a Formulation A insert, which is a coated formulation cured at 140° C. for 4 hours.

FIG. 4B depicts the in vitro drug release profile showing cumulative amount (μg) of drug released from a Formulation A insert.

FIG. 5 shows photographs of eroded Formulation A inserts taken after immersion in dissolution medium for 314 and 447 days, and the photo of the 447 day insert includes an intact insert for comparison.

FIG. 6 depicts the in vitro drug release profile for an Uncoated Formulation A insert, which is the same as Formulation A but without a coating.

FIG. 7 shows photographs of eroded Uncoated Formulation A inserts taken after immersion in dissolution medium for 287 and 352 days, and the photo of the 352 day insert includes an intact insert for comparison.

FIG. 8A depicts the in vitro drug release profile showing cumulative percent drug release from a Formulation B insert, which is a coated formulation cured at 140° C./30 minutes.

FIG. 8B depicts the in vitro drug release profile showing cumulative amount (μg) drug release from a Formulation B insert.

FIG. 9 shows photographs of eroded Formulation B inserts taken after immersion in dissolution medium for 59, 88 and 155 days.

FIG. 10 depicts the in vitro drug release profile for a Formulation C insert, an uncured coated formulation.

FIG. 11 shows photographs of two samples of eroded Formulation C inserts taken after immersion in dissolution medium for 98 days at 37° C. then 113 days at room temperature.

FIG. 12 depicts a comparison of the in vitro drug release profiles for Formulations A, B and C.

FIG. 13A depicts average amount of drug remaining in an insert versus time for an in vivo study in which inserts that had been implanted in rabbit eyes were explanted at various time points and assayed to determine the amount (μg) of vorolanib remaining in the insert. One curve shows levels for inserts from eyes in which 3 inserts were implanted, and the other shows levels for inserts from eyes in which 6 inserts were implanted.

FIG. 13B depicts cumulative percent of drug released versus time for explanted inserts from the same in vivo study. One curve shows levels for inserts from eyes in which 3 inserts were implanted, and the other shows levels for inserts from eyes in which 6 inserts were implanted.

FIG. 14 is a bar graph comparing the Corrected Total Lesion Fluorescence (CTFL) percentage change over time for different drug doses in a swine model of laser-induced choroidal neovascularization.

FIG. 15 is a graph showing the average change in best BCVA from the screening visit for the subjects in a Phase 1 clinical trial.

FIG. 16 is a graph showing the average change in CST from the screening visit for the subjects in a Phase 1 clinical trial.

FIG. 17 is a graph showing the supplemental-free rate for each visit for the subjects in a Phase 1 clinical trial.

FIG. 18 shows the results of visual acuity measurements: OKT was used to measure visual acuity. Solid bars represent mean spatial frequency threshold values, and dots show the spread of individual values within each group. The data was analyzed for significance using a 1-way ANOVA with Sidak's multiple comparison post-test for the indicated pair-wise comparison.

FIG. 19 shows the results of contrast threshold measurements: OKT was used to measure the threshold at which mice could distinguish the minimum contrast between light and dark bars presented as rotating visual stimuli. In the figure, solid bars represent mean contrast threshold values, and dots show the spread of individual values within each group. The data was analyzed for significance using a 1-way ANOVA with Sidak's multiple comparison post-test for the indicated pair-wise comparison.

FIGS. 20A-20B show retinal thickness measured by vertical Optical Coherence tomography (OCT) scans: OCT was used to obtain cross-sectional images of the retina in the vertical axis. The images were used to quantify total retinal thickness in individual eyes. A) Bars represent mean values within each study group, and dots show the spread of individual values within each group. The data was analyzed for significance using a 1-way ANOVA with Sidak's multiple comparison post-test for the indicated pair-wise comparison. B) Measurements taken at ˜3 μm increments across the entire scan, from the inferior to superior retina, are plotted on the line graph.

FIGS. 21A-21B show retinal thickness measured by horizontal OCT scans: OCT was used to obtain cross-sectional images of the retina in the horizontal axis. The images were used to quantify total retinal thickness in individual eyes. A) Bars represent mean values within each study group, and dots show the spread of individual values within each group. The data was analyzed for significance using a 1-way ANOVA with Sidak's multiple comparison post-test for the indicated pair-wise comparison. B) Measurements taken at ˜3 μm increments across the entire scan, from nasal to temporal retina, are plotted on the line graph.

FIGS. 22A-22B show Outer Nuclear Layer (ONL) thickness measured by vertical OCT scans: OCT was used to obtain cross-sectional images of the retina in the vertical axis. The images were used to quantify ONL thickness in individual eyes. A) Bars represent mean values within each study group, and dots show the spread of individual values within each group. The data was analyzed for significance using a 1-way ANOVA with Sidak's multiple comparison post-test for the indicated pair-wise comparison. B) Measurements taken at ˜3 μm increments across the entire scan, from the inferior to superior retina, are plotted on the line graph.

FIGS. 23A-23B show ONL thickness measured by horizontal OCT scans: OCT was used to obtain cross-sectional images of the retina in the horizontal axis. The images were used to quantify ONL thickness in individual eyes. A) Bars represent mean values within each study group, and dots show the spread of individual values within each group. The data was analyzed for significance using a 1-way ANOVA with Sidak's multiple comparison post-test for the indicated pair-wise comparison. B) Measurements taken at ˜3 μm increments across the entire scan, from nasal to temporal retina, are plotted on the line graph.

### DETAILED DESCRIPTION OF THE INVENTION

### 1. Active Pharmaceutical Ingredient (API)

The insert of the invention comprises the active pharmaceutical ingredient (API) vorolanib. An API is sometimes referred to as a “drug” herein. In some embodiments of the invention, the API is a pharmaceutically acceptable salt of vorolanib.

Vorolanib has the chemical designation (S,Z)—N-(1-(Dimethylcarbamoyl) pyrrolidin-3-yl)-5-((5-fluoro-2-oxoindolin-3-ylidene)methyl)-2,4-dimethyl-1H-pyrrole-3-carboxamide. Synonyms include the term “X-82”. The molecular formula is C₂₃H₂₆FN₅O₃. The solubility of vorolanib in water is less than 10 μg/mL. Vorolanib has the following structure:

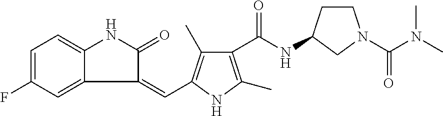 [SMILES: Cc1[nH]c(/C=C2\C(=O)Nc3ccc(F)cc32)c(C)c1C(=O)N[C@H]1CCN(C(=O)N(C)C)C1]

Vorolanib is an orally active multikinase inhibitor and can inhibit activation of vascular endothelial growth factor receptors (VEGFR) and platelet-derived growth factor receptors (PDGFR).

Methods for manufacturing vorolanib are described, e.g., in U.S. Pat. Nos. 7,683,057; 8,524,709; 8,039,470; and US Publ. Appl. No. 2019/0233403; each of which is incorporated by reference in its entirety.

As used herein, “vorolanib or a pharmaceutically acceptable salt thereof” includes amorphous and crystalline forms, polymorphs, hydrates and solvates of vorolanib or its pharmaceutically acceptable salts.

In addition, the invention contemplates the use of analogs, derivatives, pharmaceutically acceptable salts, esters, prodrugs, codrugs, and protected forms thereof of the API.

The term “pharmaceutically acceptable salt” refers to salts that retain the biological effectiveness and properties of the given compound, and which are not biologically or otherwise undesirable.

Pharmaceutically acceptable salts include salts with inorganic acids or organic acids, and salts with inorganic bases or organic bases. Those skilled in the art will recognize various synthetic methodologies that may be used to prepare nontoxic pharmaceutically acceptable salts.

Salts may be derived from inorganic acids, including hydrochloric acid, hydrobromic acid, sulfuric acid, nitric acid, phosphoric acid, and the like. Salts may be derived from organic acids, including acetic acid, propionic acid, glycolic acid, gluconic acid, pamoic acid, pyruvic acid, oxalic acid, maleic acid, malonic acid, succinic acid, fumaric acid, tartaric acid, citric acid, lactic acid, benzoic acid, cinnamic acid, mandelic acid, methanesulfonic acid, ethanesulfonic acid, p-toluene-sulfonic acid, salicylic acid, and the like.

Pharmaceutically acceptable base addition salts can be prepared from inorganic and organic bases. Salts derived from inorganic bases include sodium, potassium, lithium, ammonium, calcium and magnesium salts. Salts derived from organic bases include, but are not limited to, salts of primary, secondary and tertiary amines.

In addition, pharmaceutically acceptable salts include organic salts such as choline, glucosamine, tris, meglumine, lysine, arginine, tributylamine, and benzathine salts.

In some embodiments the API is an amorphous form, a crystalline form, a polymorph, a hydrate, or a solvate.

Unless otherwise specified, the doses described in this application (e.g., 100 μg) refer to the weight of the pharmacologically active moiety, rather than the weight of a given API salt or API ester. Thus, for example, when the insert contains a pharmaceutically acceptable salt or ester of vorolanib, the weight must be adjusted to provide an amount of the API salt that is equivalent to the amount of the API described herein. In another example, a Drug Release Rate of 100 μg/day means that the insert releases 100 μg/day of the pharmacologically active moiety (e.g., vorolanib).

Before formulation of the insert, the API may be milled to produce a fine particle size. In some embodiments, the D₉₀ for the API for use in manufacturing the insert is less than 200 μm, less than 100 μm, less than 50 μm, less than 40 μm, less than 30 μm, less than 20 μm, or less than 15 μm. In some embodiments, the D₉₀ is about 0.01 μm to about 100 μm, about 0.01 μm to about 80 μm, about 0.1 μm to about 50 μm, about 0.1 μm to about 20, about 0.1 μm to about 15 μm, about 0.1 μm to about 12 μm, about 1 μm to about 50 μm, about 1 μm to about 30 μm, about 1 μm to about 25 μm, about 1 μm to about 20 μm, about 1 μm to about 15 μm, about 1 μm to about 12 μm, about 5 μm to about 10, about 7 μm, about 8 μm, about 9 μm, about 10 μm, about 11 μm, or about 12 μm.

### 2. Ocular Drug Delivery Insert

An “ocular drug delivery insert” is a device that can be implanted in an eye, contains a drug, and can release the drug in the eye after implantation. “Ocular drug delivery insert” encompasses all of the inserts described herein.

The ocular drug delivery insert comprises a core comprising an API dispersed in a solid matrix. In some embodiments, the core is at least partially covered by a coating. In other embodiments, the insert consists only of the core. It is not surrounded by a coating or any kind of barrier surrounding the core. The insert is bioerodible.

In some embodiments, the insert comprises both a core and a coating. The coating is a layer that partially or fully surrounds the core. The coating is an outer layer, which may be preformed into the desired shape (e.g., it may be a tube) before it is placed around the core, or the coating may be formed, e.g., by coextrusion of core and coating, spraying the coating onto the core, or dipping the core into the coating material once or multiple times (e.g., 1-10 coats). If the core is coated, the coating may completely surround the core, or may only partially surround the core.

The insert may be a variety of different shapes, e.g., a cylinder, rod, sphere, or disk. In some embodiments, the insert is cylindrical in shape, and the coating covers the entire surface of the cylinder except the ends of the rod or cylinder. The ends of the rod may act as delivery ports. In some embodiments, one end of the cylinder is covered by the coating and the other is not. In some embodiments, one of the ends is coved by a drug-impermeable cap such as a silicone cap.

A rod is a solid geometrical figure with parallel sides, wherein the length of a side is longer than the diameter or longest side of the shape of the cross section. The cross-section shape may be a circle, oval, square, rectangle, triangle, or polygon such as a hexagon. One of skill in the art will recognize that due to manufacturing processes, the insert shape may not be precise, e.g., the exterior may not be smooth and perfectly even. For example, the sides of the cylinder or rod may not be perfectly straight or perfectly parallel. A cross section of a cylinder or rod may not be a perfect circle or oval. Cross sections of other shapes may not precisely meet the definition of those shapes. For example, a square cross section may not have perfectly straight sides and the angles of the corners may not be exactly 90 degrees. Spheres or pellets may not be perfectly spherical.

a. Matrix

In some embodiments, the core is a solid matrix comprising a matrix polymer and an API, which may be present in a solid form, such as a powder, particles, or granules, dispersed throughout the matrix. The matrix ingredients and API form a homogenous mixture in which the API is dispersed. The matrix is solid at room temperature and is bioerodible. The matrix helps to control the rate of release of the API, thus modifying the rate of API release as compared to unformulated API. In some embodiments, the matrix slows the rate of drug release and provides for prolonged delivery of the drug, and less frequent dosing.

In some embodiments, the matrix also comprises other pharmaceutically acceptable ingredients. In other embodiments, the only material used to form the matrix is one or more matrix polymers.

The polymer used to form the matrix (the “matrix polymer”) may comprise one or more of the following: polyvinyl alcohol (PVA), poly(caprolactone) (PCL), polyethylene glycol (PEG), poly(dl-lactide-co-glycolide) (PLGA), polyvinyl alcohol (PVA), poly(lactic acid) (PLA), poly(glycolic acid) (PGA), polyalkyl cyanoacrylate, or a copolymer thereof.

In certain embodiments, the matrix polymer comprises PVA. In some embodiments, the only inactive pharmaceutical ingredient in the matrix is PVA.

Various grades of PVA may be used. The degree of hydrolysis (DH) of the PVA may be about 70% to about 99⁺%, and the molecular weight (MW) may be about 6000-200,000, i.e., the matrix polymer is about 70 mole % to about 99⁺ mole % hydrolyzed PVA having a molecular weight of about 6,000-200,000. For example, the DH may be about 70% to about 80%, about 80% to about 90%, about 80% to about 85%, about 88% to about 90%, about 90% to about 99⁺%, about 98 to about 99⁺%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99⁺%; and the MW may be about 5000, about 6000, about 7000, about 8000, about 9000, about 10,000, about 15,000, about 18,000, about 20,000, about 25,000, about 30,000, about 40,000, about 50,000, about 60,000, about 70,000, about 75,000, about 78,000, about 80,000, about 85,000, about 90,000, about 100,000, about 108,000, about 110,000, about 120,000, about 125,000, about 130,000, about 133,000, about 140,000, about 146,000, about 150,000, about 160,000, about 170,000, about 180,000, about 186,000, about 190,000 or about 200,000. In some embodiments, the MW may be about 5000-10,000, about 6000-10,000, about 9000-10,000, about 10,000-30,000, about 10,000-25,000, about 25,000-50,000, about 30,000-70,000, about 60,000-80,000, about 70,000-80,000, about 75,000-80,000, about 75,000-100,000, about 89,000-98,000, about 85,000-124,000, about 100,000-150,000, about 146,000-186,000, or about 150,000-200,000. In certain embodiments the PVA is MW 6,000, 80% hydrolyzed, MW 9,000-10,000, 80% hydrolyzed, MW 25,000, 88% hydrolyzed, MW 25,000, 98% hydrolyzed, MW 30,000-70,000, 87-90% hydrolyzed, MW 78,000, 88% hydrolyzed, MW 78,000, 98% hydrolyzed, MW 78,000, 99+% hydrolyzed, MW 89,000-98,000, 99+% hydrolyzed, MW 85,000-124,000, 87-89% hydrolyzed, MW 108,000, 99⁺% hydrolyzed, MW 125,000, 88% hydrolyzed, MW 133,000, 99% hydrolyzed, or MW 146,000-186,000, 99+% hydrolyzed.

In other embodiments, the matrix polymer comprises a mixture of two, three or four different grades of PVA. In some embodiments the PVA is a mixture of two different grades of PVA. In some embodiments, the ratio of the two grades in the mixture is from 1:1 to 1:15. In some embodiments, the ratio of the two grades is 1:6, 1:7, 1:8, 1:9, 1:10, 1:11 or 1:12 of the slower eroding PVA to the faster eroding PVA. The PVA erosion rate may be measured as described in Example 1. For example, in some embodiments the mixture of PVA has a ratio of 1:9 6000 MW, 80% DH to 125,000 MW, 88% DH. In other embodiments, the ratio of the two grades in the mixture is from 1:1 to 1:15, e.g., 1:6, 1:7, 1:8, 1:9, 1:10, 1:11 or 1:12, of the faster eroding PVA to the slower eroding PVA.

Examples of PVA mixtures include a mixture of MW 6,000, 80% hydrolyzed with MW 78,000, 98% hydrolyzed; a mixture of MW 6,000, 80% hydrolyzed with MW 78,000, 99⁺% hydrolyzed; a mixture of MW 78,000, 98% hydrolyzed with MW 78,000, 99⁺% hydrolyzed; and a mixture of MW 6,000, 80% hydrolyzed with MW 125,000, 88% hydrolyzed.

The MW and DH should be selected to provide the rate of drug release desired for the particular drug, the indication for which the ocular drug delivery insert will be used, the duration of drug release desired and the rate of erosion desired.

The polymer solution used to form the core matrix may comprise about 1% w/w to about 20% w/w, about 1% w/w to about 15% w/w, about 2% w/w to about 15% w/w, about 2% w/w to about 12% w/w, about 2% w/w to about 10% w/w, about 3% w/w to about 10% w/w, about 3% w/w to about 8% w/w, about 3% w/w to about 6% w/w, about 5% w/w to about 20% w/w, about 5% w/w to about 15% w/w, about 10% w/w to about 20% w/w, about 5% w/w to about 8% w/w, about 5% w/w to about 7% w/w, about 6% w/w to about 8% w/w, about 6% w/w to about 7% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, about 11% w/w, about 11.5% w/w, about 12% w/w, about 13% w/w, about 14% w/w or about 15% w/w polymer, such as PVA, in a solvent, such as water or ethanol.

The polymer solution and the API may be combined in a ratio of, e.g., about 0.5:1, about 1:1, about 1:1.2, about 1:1.5, about 1:1.7, or about 1:2 w/w API:polymer solution.

In some embodiments the core comprises vorolanib or a pharmaceutically acceptable salt thereof and PVA. In some embodiments the core consists of vorolanib or a pharmaceutically acceptable salt thereof and PVA.

In further embodiments, the PVA solution and API are combined in a ratio of about 1:1 w/w API:PVA solution.

In some embodiments, the PVA solution and API are combined in a ratio of about 1:2 w/w API:PVA solution.

In some embodiments, the core comprises about 0.1% w/w to about 90% w/w, about 0.1% w/w to about 80% w/w, about 0.1% w/w to about 70% w/w, about 0.1% w/w to about 60% w/w, about 0.1% w/w to about 50% w/w, about 0.1% w/w to about 40% w/w, about 0.1% w/w to about 30% w/w, about 0.1% w/w to about 25% w/w, about 0.1% w/w to about 20% w/w, about 0.1% w/w to about 15% w/w, about 0.1% w/w to about 10% w/w, about 1% w/w to about 20%, about 1% w/w to about 15%, about 1% w/w to about 10% w/w, about 1% w/w to about 9% w/w, about 1% w/w to about 8% w/w, about 1% w/w to about 7% w/w, about 1% w/w to about 6% w/w, about 2% w/w to about 10% w/w, about 2% w/w to about 8% w/w, about 2% w/w to about 6% w/w, about 3% w/w to about 90% w/w, about 3% w/w to about 75% w/w, about 3% w/w to about 60% w/w, about 3% w/w to about 40% w/w, about 3% w/w to about 20% w/w, about 3% w/w to about 15% w/w, about 3% w/w to about 10% w/w, about 3% w/w to about 8% w/w, about 3% w/w to about 7, about 3% w/w to about 5% w/w, about 4% w/w to about 60% w/w, about 4% w/w to about 50% w/w, about 4% w/w to about 40% w/w, about 4% w/w to about 25% w/w, about 4% w/w to about 20% w/w, about 4% w/w to about 15% w/w, about 4% w/w to about 10% w/w, about 4% w/w to about 8% w/w, about 4% w/w to about 7% w/w, about 5% w/w to about 10% w/w, about 5% w/w to about 8% w/w, or about 5% w/w to about 7% w/w inactive (non-API) ingredients, such as a matrix polymer. These weight percentages are based on the dry weight of the core (i.e., after any drying steps in processing).

In some embodiments, the amount of matrix polymer in the core is about 0.1% w/w to about 90% w/w, about 0.1% w/w to about 80% w/w, about 0.1% w/w to about 70% w/w, about 0.1% w/w to about 60% w/w, about 0.1% w/w to about 50% w/w, about 0.1% w/w to about 40% w/w, about 0.1% w/w to about 30% w/w, about 0.1% w/w to about 25% w/w, about 0.1% w/w to about 20% w/w, about 0.1% w/w to about 15% w/w, about 0.1% w/w to about 10% w/w, about 1% w/w to about 20%, about 1% w/w to about 15%, about 1% w/w to about 10% w/w, about 1% w/w to about 9% w/w, about 1% w/w to about 8% w/w, about 1% w/w to about 7% w/w, about 1% w/w to about 6% w/w, about 2% w/w to about 10% w/w, about 2% w/w to about 8% w/w, about 2% w/w to about 6% w/w, about 3% w/w to about 90% w/w, about 3% w/w to about 75% w/w, about 3% w/w to about 60% w/w, about 3% w/w to about 40% w/w, about 3% w/w to about 20% w/w, about 3% w/w to about 15% w/w, about 3% w/w to about 10% w/w, about 3% w/w to about 8% w/w, about 3% w/w to about 7, about 3% w/w to about 5% w/w, about 4% w/w to about 60% w/w, about 4% w/w to about 50% w/w, about 4% w/w to about 40% w/w, about 4% w/w to about 25% w/w, about 4% w/w to about 20% w/w, about 4% w/w to about 15% w/w, about 4% w/w to about 10% w/w, about 4% w/w to about 8% w/w, about 4% w/w to about 7% w/w, about 5% w/w to about 10% w/w, about 5% w/w to about 8% w/w, or about 5% w/w to about 7% w/w; or is about 1% w/w, 1.5% w/w, 2% w/w, 2.5% w/w, 3% w/w, 3.5% w/w, 4% w/w, 4.5% w/w, 5% w/w, 5.5% w/w, 6% w/w, 6.5% w/w, 7% w/w, 7.5% w/w, 8% w/w, 8.5% w/w, 9% w/w, 9.5% w/w, 10% w/w, 10.5% w/w, 11% w/w, 11.5% w/w, 12% w/w, 15% w/w, 18% w/w, 20% w/w, 25% w/w, 30% w/w, 35% w/w, 40% w/w, 50% w/w, 55% w/w, 60% w/w, 65% w/w, or 70% w/w. These weight percentages are based on the dry weight of the core (i.e., after any drying steps in processing).

The term “the insert consists of” a core comprising a solid matrix and API means that the entire insert is in the form of a solid matrix and API. The matrix may also include additional ingredients, but the insert does not have a shell, coating, cap, covering or tube or other outer layer, so that when immersed in a fluid environment, such as the vitreous humor of the eye or an in vitro drug release medium, the exterior of the core is in direct contact with this fluid.

b. Coating

In some embodiments of the invention, the insert comprises or consists of (a) a core comprising an API and a solid matrix, and (b) a coating. In other embodiments, the insert does not comprise a coating.

In some embodiments, the coating is permeable to the passage of the API and acts as a diffusion membrane for the active pharmaceutical ingredient. A diffusion membrane may modify the API release rate of the matrix. The diffusion membrane may operate by, for example, modifying fluid flow into the matrix and/or limiting the passage of the API out of the matrix. In other embodiments, the coating increases the durability of the insert, as compared to an uncoated core, e.g., during processing, packaging, and/or delivering the dose. In certain embodiments, the coating both modifies the API release rate and increases the durability of the insert.

The coating may completely surround the core or may only partially surround the core. In some embodiments, the coating substantially covers the core, which means that it covers at least 70% of the surface area of the core. In some embodiments, the coating covers at least 75%, at least 80%, at least 85%, at least 90%, or at least 95% of the surface area of the core. In other embodiments, the coating surrounds about 40% to about 98%, about 50% to about 98%, about 60% to about 98%, about 70% to about 95%, about 70% to about 98%, about 70% to about 100%, about 80% to about 95%, about 80% to about 96%, about 80% to about 98%, about 80% to about 99%, about 90% to about 99%, or about 90% to about 98% of the surface area of the core. For a cylindrical insert, the surface area A is calculated as A=2πrL+2πr2, where r is the radius and L is the length of the insert. In some embodiments, an area of the core is left uncovered by a coating to form a delivery port. In some embodiments, more than one area is left uncovered to form more than one delivery port.

The delivery port is permeable to the API.

In some embodiments, the insert is rod-shaped, e.g., cylindrical, and only the two ends of the rod/cylinder are uncoated.

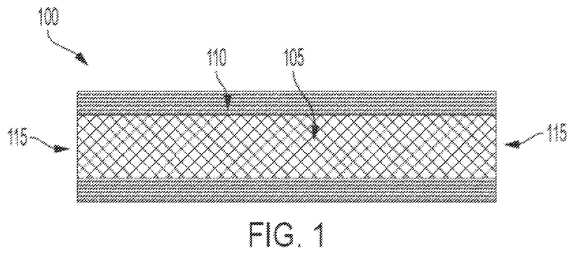

To provide an illustration of an embodiment of the ocular drug delivery insert of the invention, FIG. 1 shows a longitudinal cross-sectional view of an ocular drug delivery insert 100 according to one embodiment of the invention. Insert 100 comprises solid matrix core 105. Insert 100 further comprises a coating 110, substantially surrounding the core 105. Insert 100 also features two delivery ports 115 which are located at opposite ends of insert 100. In this particular embodiment, at least one of the delivery ports 115 comprises a membrane permeable to the API contained in core 105 to allow the API to be released from the delivery port/s 115.

In some embodiments, like the matrix, the coating is bioerodible.

The coating may comprise polymeric and/or nonpolymeric ingredients. In some embodiments, the coating comprises one or more polymers such as polyvinyl alcohol (PVA), poly(caprolactone) (PCL), polyethylene glycol (PEG), poly(dl-lactide-co-glycolide) (PLGA), poly(lactic acid) (PLA), poly(glycolic acid) (PGA), polyalkyl cyanoacrylate, or a copolymer thereof.

In embodiments in which the core is coated, the coating may be formed from 1-10 coats of polymer. For example, the core may be coated with 1 coat, 2 coats, 3 coats, 4 coats, 5 coats, 6 coats, 7 coats, 8 coats, 9 coats, or 10 coats. In some embodiments, each of the coats comprise the same polymer as the other coats. In certain embodiments, each of the coats consists of the same polymer as the other coats. In other embodiments in which the coating is formed from more than one coat, at least two of the coats comprise different polymers.

In certain embodiments, the coating comprises PVA. In other embodiments, the coating consists of PVA. In some embodiments, the only inactive pharmaceutical ingredient in the coating is PVA. In other embodiments, both the matrix polymer comprises PVA and the coating comprises PVA. In yet other embodiments, both the matrix polymer consists of PVA and the coating consists of PVA.

Various grades of PVA may be used. The degree of hydrolysis (DH) of the PVA may be about 70% to about 99%, and the molecular weight (MW) may be about 6000-200,000, i.e., the matrix polymer is about 70 mole % to about 99⁺ mole % hydrolyzed PVA having a molecular weight of about 6,000-200,000. For example, the DH may be about 70% to about 80%, about 80% to about 90%, about 80% to about 85%, about 88% to about 90%, about 90% to about 99⁺%, about 98 to about 99%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%; and the MW may be about 5000, about 6000, about 7000, about 8000, about 9000, about 10,000, about 15,000, about 18,000, about 20,000, about 25,000, about 30,000, about 40,000, about 50,000, about 60,000, about 70,000, about 75,000, about 78,000, about 80,000, about 85,000, about 90,000, about 100,000, about 108,000, about 110,000, about 120,000, about 125,000, about 130,000, about 133,000, about 140,000, about 146,000, about 150,000, about 160,000, about 170,000, about 180,000, about 186,000, about 190,000 or about 200,000. In some embodiments, the MW may be about 5000-10,000, about 6000-10,000, about 9000-10,000, about 10,000-30,000, about 10,000-25,000, about 25,000-50,000, about 30,000-70,000, about 60,000-80,000, about 70,000-80,000, about 75,000-80,000, about 75,000-100,000, about 89,000-98,000, about 85,000-124,000, about 100,000-150,000, about 146,000-186,000, or about 150,000-200,000. In certain embodiments the PVA is MW 6,000, 80% hydrolyzed, MW 9,000-10,000, 80% hydrolyzed, MW 25,000, 88% hydrolyzed, MW 25,000, 98% hydrolyzed, MW 30,000-70,000, 87-90% hydrolyzed, MW 78,000, 88% hydrolyzed, MW 78,000, 98% hydrolyzed, MW 78,000, 99+% hydrolyzed, MW 89,000-98,000, 99+% hydrolyzed, MW 85,000-124,000, 87-89% hydrolyzed, MW 108,000, 99⁺% hydrolyzed, MW 125,000, 88% hydrolyzed, MW 133,000, 99% hydrolyzed, or MW 146,000-186,000, 99+% hydrolyzed.

In other embodiments, the PVA is a mixture of two, three or four different grades of PVA. In some embodiments the PVA is a mixture of two different grades of PVA. In some embodiments, the ratio of the two grades in the mixture is from 1:1 to 1:15. In some embodiments, the ratio of the two grades is 1:6, 1:7, 1:8, 1:9, 1:10, 1:11 or 1:12 of the slower eroding PVA to the faster eroding PVA. The PVA erosion rate may be measured as described in Example 1. For example, in some embodiments the mixture of PVA has a ratio of 1:9 6000 MW, 80% DH to 125,000 MW, 88% DH. In other embodiments, the ratio of the two grades in the mixture is from 1:1 to 1:15, e.g., 1:6, 1:7, 1:8, 1:9, 1:10, 1:11 or 1:12, of the faster eroding PVA to the slower eroding PVA.

Examples of PVA mixtures include a mixture of MW 6,000, 80% hydrolyzed with MW 78,000, 98% hydrolyzed; a mixture of MW 6,000, 80% hydrolyzed with MW 78,000, 88% hydrolyzed; a mixture of MW 6,000, 80% hydrolyzed with MW 78,000, 99% hydrolyzed; a mixture of MW 78,000, 88% hydrolyzed with MW 78,000, 98% hydrolyzed; a mixture of MW 78,000, 98% hydrolyzed with MW 78,000, 99% hydrolyzed; and a mixture of MW 6,000, 80% hydrolyzed with MW 125,000, 88% hydrolyzed.

In certain embodiments, the core comprises a mixture of two different grades of PVA. In some embodiments, the coating comprises a mixture of two different grades of PVA. In yet other embodiments, both the core and coating comprise a mixture of two different grades of PVA. Where the coating comprises more than one coat of PVA, one or more of the coats may comprise a mixture of two different grades of PVA.

In embodiments in which both the core and coating comprise PVA, the core PVA and the coating PVA may be the same or different grades of PVA. Used herein, the term “different grade of PVA” means the PVA differs in molecular weight (MW), degree of hydrolysis (DH) or both MW and DH. In addition, as used herein, a mixture of grades of PVA is a “different grade of PVA” if the PVA to which the mixture is compared is not a mixture of the same exact PVA grades, e.g., a mixture of 6,000, 80% hydrolyzed PVA with MW 78,000, 98% hydrolyzed PVA, would be considered a different grade of PVA from a PVA composition that contains only MW 78,000, 98% hydrolyzed PVA, or that contains a mixture of MW 6,000, 80% hydrolyzed PVA with MW 125,000, 88% hydrolyzed PVA.

Thus, the core PVA and the coating PVA may have the same MW and DH, or may differ in MW or DH, or may differ in both MW and DH. In some embodiments, the core comprises PVA, and the insert comprises a coating comprising PVA, wherein the MW of the coating PVA is the same as the MW of the core PVA, and the DH of the coating PVA is lower than the DH of the core PVA. In some embodiments, the MW and the DH of the coating PVA are each lower than the MW and DH of the core PVA.

In some embodiments, the coating is formed from more than one coat. When the insert coating comprises more than one coat of PVA, PVA having the same MW and DH may be used for the core and at least one of the coat/s. In other embodiments, the core comprises a PVA that differs in MW and/or DH from the PVA in at least one coat. In some embodiments, the core comprises a PVA that differs in both MW and DH from the PVA in at least one coat. In other embodiments, the PVA in the core and the PVA in at least one coat have the same MW but differ in DH. In some embodiments the DH of the PVA in at least one coat is lower than the DH of the PVA in the core. In other embodiments, the PVA in the core and the PVA in at least one coat differ in MW but have the same DH. In some embodiments the MW of the PVA in at least one coat is lower than the MW of the PVA in the core

In some embodiments, the insert coating comprises a single coat comprising PVA. In other embodiments, the insert coating comprises more than one coat comprising PVA, and the PVA in each coating has the same MW and DH. In some embodiments, at least one coat comprises PVA that differs in MW and/or DH from the PVA in at least one other coat. In some embodiments, at least one coat comprises PVA that differs in both MW and DH from the PVA in at least one other coat. In some embodiments, no two coats comprise the same grade PVA, i.e., the PVA in each coat differs in MW and/or DH from each of the other coats.

In some embodiments in which the insert coating comprises more than one coat comprising PVA, the PVA in the outermost coat is more soluble (in PBS) than the PVA in any of the other coats. In some embodiments, the PVA in at least one of the coats is more soluble than the core PVA.

In certain embodiments, the insert comprises (a) a solid matrix core comprising PVA and an API, and (b) a coating comprising PVA substantially surrounding the core; and the DH of the PVA in the coating is lower than the DH of the PVA in the core. In one embodiment of this insert, the insert comprises 2 coats comprising PVA. In other embodiments, the insert comprises 3 coats comprising PVA. In yet other embodiments, the insert comprises 4 coats comprising PVA. In further embodiments, the insert comprises 5 coats comprising PVA. In yet other embodiments, the insert comprises 6 coats comprising PVA.

In embodiments with more than one coat, the first coat applied to the core is the innermost coat, and the last coat applied is the outermost coat. In some embodiments of these inserts having more than one coat of PVA, the DH of the PVA in the innermost coat is higher than the DH of the PVA in the outermost coat. In other embodiments of these inserts having more than one coat of PVA, the MW of the PVA in the innermost coat is higher than the MW of the PVA in the outermost coat. In some embodiments, the DH of the PVA in the outermost coat is lower than the DH of the PVA in each of the other coats. In other embodiments, the MW and DH of the PVA in the outermost coat is lower than the MW and DH of the PVA in any of the other coats.

In some aspects, the insert comprises (a) a solid matrix core comprising a PVA selected from the group consisting of MW 6,000, 80% hydrolyzed, MW 9,000-10,000, 80% hydrolyzed, MW 25,000, 88% hydrolyzed, MW 25,000, 98% hydrolyzed, MW 30,000-70,000, 87-90% hydrolyzed, MW 78,000, 88% hydrolyzed, MW 78,000, 98% hydrolyzed, MW 78,000, 99+% hydrolyzed, MW 89,000-98,000, 99+% hydrolyzed, MW 85,000-124,000, 87-89% hydrolyzed, MW 108,000, 99⁺% hydrolyzed, MW 125,000, 88% hydrolyzed, MW 133,000, 99% hydrolyzed, MW 146,000-186,000, 99+% hydrolyzed, and mixtures thereof; and an API, and (b) at least one coating comprising PVA substantially surrounding the core, wherein the PVA in the coating is selected from a PVA selected from the group consisting of MW 6,000, 80% hydrolyzed, MW 9,000-10,000, 80% hydrolyzed, MW 25,000, 88% hydrolyzed, MW 25,000, 98% hydrolyzed, MW 30,000-70,000, 87-90% hydrolyzed, MW 78,000, 88% hydrolyzed, MW 78,000, 98% hydrolyzed, MW 78,000, 99-% hydrolyzed, MW 89,000-98,000, 99+% hydrolyzed, MW 85,000-124,000, 87-89% hydrolyzed, MW 108,000, 99% hydrolyzed, MW 125,000, 88% hydrolyzed, MW 133,000, 99% hydrolyzed, MW 146,000-186,000, 99+% hydrolyzed, and mixtures thereof; and wherein the PVA in the core and the PVA in at least one coating are different grades of PVA. In other aspects if this inert, the insert comprises at least 2 coats of PVA and the DH of the PVA in the outermost coat is lower than the DH of any of the PVA in each of the other coats.

The invention provides the ability to tailor the PVA grades used to manufacture the ocular insert. The PVA MW and DH of core and coating should be selected to provide the rate of drug release desired for the particular drug, the indication for which the ocular insert will be used, the duration of drug release desired, and the rate of erosion desired. Different durations of drug release may be desired for different ocular diseases or conditions. For example, a 12 month duration (such as is provided by Formulation A) of drug release may be desirable for the treatment of diabetic retinopathy, whereas a duration of less than a month may be desirable for an insert for inhibiting ocular inflammation caused by injury or surgery.

The polymer solution used to form the coating may comprise about 1% w/w to about 20% w/w, about 1% w/w to about 15% w/w, about 1% w/w to about 10% w/w, about 2% w/w to about 15% w/w, about 2% w/w to about 12% w/w, about 2% w/w to about 10% w/w, about 2% w/w to about 8% w/w, about 2% w/w to about 6% w/w, about 3% w/w to about 10% w/w, about 3% w/w to about 8% w/w, about 3% w/w to about 6% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w about 10.5% w/w, about 11% w/w, about 11.5% w/w, about 12% w/w, about 13% w/w, about 14% w/w or about 15% w/w polymer, such as PVA, in a solvent, such as water or ethanol.

For inserts comprising a PVA coating, the core may be covered with 1-10 coats of a solution of PVA, i.e., the insert may comprise 1-10 PVA coatings. For example, the insert may comprise 1 coat, 2 coats, 3 coats, 4 coats, 5 coats, 6 coats, 7 coats, 8 coats, 9 coats, or 10 coats of PVA.

In some embodiments, the weight of the insert coating is about 0.1% w/w to about 60% w/w, about 0.1% w/w to about 40% w/w, about 0.1% w/w to about 20% w/w, about 1% w/w to about 40% w/w, about 1% w/w to about 30% w/w, about 1% w/w to about 20% w/w, about 1% w/w to about 10% w/w, about 1% w/w to about 6% w/w, about 3% w/w to about 20% w/w, about 3% w/w to about 10% w/w, about 3% w/w to about 6% w/w, about 5% w/w to about 30% w/w, about 5% w/w to about 25% w/w, about 5% w/w to about 20% w/w, about 5% w/w to about 15% w/w, about 5% w/w to about 10% w/w, about 10% w/w to about 25% w/w, about 10% w/w to about 20% w/w, about 10% w/w to about 18% w/w, or about 12% w/w to about 18% w/w of the insert. These weight percentages are based on the dry weight of the insert (i.e., after any drying steps in processing).

In some embodiments, the total amount of inactive ingredients in the insert is about 0.1% w/w to about 90% w/w, about 0.1% w/w to about 80% w/w, about 0.1% w/w to about 70% w/w, about 0.1% w/w to about 60% w/w, about 0.1% w/w to about 50% w/w, about 0.1% w/w to about 40% w/w, about 0.1% w/w to about 30% w/w, about 0.1% w/w to about 25% w/w, about 0.1% w/w to about 20% w/w, about 0.1% w/w to about 15% w/w, about 0.1% w/w to about 10% w/w, about 1% w/w to about 70% w/w, about 1% w/w to about 50% w/w, about 1% w/w to about 20%, about 1% w/w to about 15%, about 1% w/w to about 10% w/w, about 1% w/w to about 9% w/w, about 1% w/w to about 8% w/w, about 1% w/w to about 7% w/w, about 1% w/w to about 6% w/w, about 2% w/w to about 10% w/w, about 2% w/w to about 8% w/w, about 2% w/w to about 6% w/w, about 3% w/w to about 90% w/w, about 3% w/w to about 75% w/w, about 3% w/w to about 60% w/w, about 3% w/w to about 40% w/w, about 3% w/w to about 20% w/w, about 3% w/w to about 15% w/w, about 3% w/w to about 10% w/w, about 3% w/w to about 8% w/w, about 3% w/w to about 7, about 3% w/w to about 5% w/w, about 4% w/w to about 60% w/w, about 4% w/w to about 50% w/w, about 4% w/w to about 40% w/w, about 4% w/w to about 25% w/w, about 4% w/w to about 20% w/w, about 4% w/w to about 15% w/w, about 4% w/w to about 10% w/w, about 4% w/w to about 8% w/w, about 4% w/w to about 7% w/w, about 5% w/w to about 40% w/w, about 5% w/w to about 30% w/w, about 5% w/w to about 25% w/w, about 5% w/w to about 20% w/w, about 5% w/w to about 15% w/w, about 5% w/w to about 7% w/w, about 10% w/w to about 25% w/w, about 10% w/w to about 22% w/w, about 15% w/w to about 25% w/w, about 15% w/w to about 22% w/w, or about 18% w/w to about 22% w/w. These weight percentages are based on the dry weight of the insert (i.e., after any drying steps in processing).

In some embodiments, the amount of PVA in the insert is about 0.1% w/w to about 30% w/w, about 0.1% w/w to about 25% w/w, about 0.1% w/w to about 20% w/w, about 0.1% w/w to about 15% w/w, about 0.1% w/w to about 10% w/w, about 1% w/w to about 80% w/w, about 1% w/w to about 75% w/w, about 1% w/w to about 60% w/w, about 1% w/w to about 30% w/w, about 1% w/w to about 20%, about 1% w/w to about 15%, about 1% w/w to about 10% w/w, about 1% w/w to about 9% w/w, about 1% w/w to about 8% w/w, about 1% w/w to about 7% w/w, about 1% w/w to about 6% w/w, about 2% w/w to about 10% w/w, about 2% w/w to about 8% w/w, about 2% w/w to about 6% w/w, about 3% w/w to about 90% w/w, about 3% w/w to about 80% w/w, about 3% w/w to about 75% w/w, about 3% w/w to about 70% w/w, about 3% w/w to about 60% w/w, about 3% w/w to about 40% w/w, about 3% w/w to about 20% w/w, about 3% w/w to about 15% w/w, about 3% w/w to about 10% w/w, about 3% w/w to about 8% w/w, about 3% w/w to about 7, about 3% w/w to about 5% w/w, about 4% w/w to about 60% w/w, about 4% w/w to about 50% w/w, about 4% w/w to about 40% w/w, about 4% w/w to about 25% w/w, about 4% w/w to about 20% w/w, about 4% w/w to about 15% w/w, about 4% w/w to about 10% w/w, about 4% w/w to about 8% w/w, about 4% w/w to about 7% w/w, about 5% w/w to about 40% w/w, about 5% w/w to about 30% w/w, about 5% w/w to about 25% w/w, about 5% w/w to about 20% w/w, about 5% w/w to about 15% w/w, about 5% w/w to about 10% w/w, about 5% w/w to about 7% w/w, about 10% w/w to about 25% w/w, about 10% w/w to about 22% w/w, about 15% w/w to about 25% w/w, about 15% w/w to about 22% w/w, or about 18% w/w to about 22% w/w. These weight percentages are based on the dry weight of the insert (i.e., after any drying steps in processing).

In some embodiments, the invention provides an insert having a very high drug content, relative to the inactive ingredients in the insert, which is surprising given the ability of the insert to provide release of the drug over extended periods. In some embodiments, the amount of API in the insert is about 5% w/w to about 98% w/w, about 10% w/w to about 98% w/w, about 15% w/w to about 98% w/w, about 20% w/w to about 98% w/w, about 30% w/w to about 98% w/w, about 40% w/w to about 98% w/w, about 50% w/w to about 98% w/w, about 60% w/w to about 98% w/w, about 65% w/w to about 98% w/w, about 70% w/w to about 98% w/w, about 75% w/w to about 98% w/w, about 65% w/w to about 90% w/w, about 70% w/w to about 90% w/w, about 75% w/w to about 90% w/w, about 80% w/w to about 90% w/w, about 80% w/w to about 99% w/w, about 85% w/w to about 98% w/w, about 85% w/w to about 99% w/w, about 90% w/w to about 99% w/w or about 90% w/w to about 98% w/w. These weight percentages are based on the dry weight of the insert (i.e., after any drying steps in processing).

In some embodiments, the only inactive ingredient in the insert is a polymer such as PVA.

The thickness of the coat around the core may be e.g., about 20 μm to about 400 μm, about 20 μm to about 300 μm, about 20 μm to about 200 μm, about 20 μm to about 100 μm, about 5 μm to about 75 μm, about 5 μm to about 50 μm, or about 5 μm to about 25 μm.

c. Insert Shape and Dimensions

In some embodiments, when the insert is prepared for implantation within the vitreous of the eye, the insert does not exceed about 15 mm, or preferably does not exceed about 10 mm, in any direction, so that the insert can be inserted through an incision of 15 mm or smaller.

In some embodiments, the insert may be shaped and sized for injection. In some embodiments, the insert is sized and shaped to fit through a cannula or needle of 20 gauge or smaller. This means that the insert can be injected through either a cannula or a needle having the recited gauge without an unusual amount of force. The phrase “or smaller” in this context means having a smaller outer diameter. A smaller outer diameter will correspond to a larger gauge size number, e.g., a 25 gauge needle has a smaller outer diameter than a 22 gauge needle.

In some embodiments, the insert is sized and shaped to fit through a 20 to 27 gauge needle or cannula, a 21 to 27 gauge needle or cannula, a 22 to 27 gauge needle or cannula, a 23 to 27 gauge needle or cannula, a 24 to 27 gauge needle or cannula, a 25 to 27 gauge needle or cannula, or a 25.5 to 27 gauge needle or cannula.

In other embodiments, the insert is sized and shaped to fit through a cannula or needle of 20 gauge or smaller, 22 gauge or smaller, 23 gauge or smaller, 24 gauge or smaller, 25 gauge or smaller, 25.5 gauge or smaller, 26 gauge or smaller, or 26.5 gauge or smaller. Preferably, the insert is sized and shaped to fit through a cannula or needle smaller than 25 gauge, smaller than 26 gauge, or smaller than 27 gauge. In some embodiments, the insert is sized and shaped to fit through a cannula or needle of about 29 gauge to about 25.5 gauge, such as from about 28 gauge to about 25.5 gauge, or from about 28 gauge to about 26 gauge. In some embodiments, the needle or cannula is about 22, 22s, 23, 24 or 25 gauge, but preferably is about 25.5, 26, 26.5, 26s, 27, 27.5, 28, 28.5, 29, 29.5, 30 or 30.5 gauge.

In some embodiments the insert is rod-shaped, cylindrical or spherical, and may be less than about 12 mm long and less than about 1 mm in diameter.

In some embodiments, the insert may be rod shaped or cylindrical and does not exceed 8 mm in length and 3 mm in diameter.

In some embodiments, the insert has a length of about 1 mm to 10 mm, 2 mm to 10 mm, 1 mm to 4 mm, 4 mm to 8 mm, 6 mm to 10 mm, 8 mm to 10 mm, 1 mm to 12 mm, 2 mm to 12 mm, or 4 mm to 12 mm; about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, or about 15 mm.

In some embodiments, the insert has a diameter of about 0.1 mm to about 2 mm, about 0.1 mm to about 1 mm, about 0.1 mm to about 0.8 mm, about 0.1 mm to about 0.6 mm, about 0.1 mm to about 0.5 mm, about 0.3 mm to about 0.5 mm, about 0.3 mm to about 0.4 mm, about 0.2 mm to 0.4 mm, about 0.1 mm to 0.2 mm, or about 0.4 mm to about 0.6 mm; about 0.57 mm, about 0.50 mm, about 0.41 mm, about 0.42 mm, about 0.37 mm, about 0.34 mm, about 0.31 mm, about 0.26 mm, or about 0.15 mm.

d. Insert Manufacture

The insert may be manufactured by mixing the API with a matrix polymer. In some embodiments, the matrix polymer is a solution of 1 or more polymers in a solvent, e.g., in water or ethanol. The API, matrix polymer solution, and any other matrix ingredients are mixed to form a paste suitable for extrusion through a dispensing tip. The paste may be extruded through an 18-25 gauge cannula or dispensing tip. In some embodiments a 21-23 or a 23-26 gauge cannula or dispensing tip is used. For example, the gauge of the cannula or dispensing tip may be 20, 21, 22, 23, 24, 25 or 26. The extruded paste is referred to herein as an extrudate, an elongated shaped matrix, or a rod. The rods may be about 4-5 inches (about 10-13 cm) in length. The extrudate is solid at room temperature. The extrudate may be coated with one or more additional layers. In some embodiments, the extrudate is dried at room temperature for at least 24 hours before coating.

In an extrusion process, extrusion parameters may be controlled, such as fluid pressure, flow rate, and temperature of the material being extruded. Suitable extruders may be selected for the ability to deliver the co-extruded materials at pressures and flow rates sufficient to form the product at sizes of the die head and exit port or dispensing tip that will produce a product which, when segmented and dried, can be injected through a needle or cannula as described herein.

If a polymer solution was used, and the extrudates are to be coated, the extruded API-polymer mixture is allowed to dry before coating. For example, the extrudate may be allowed to dry for about 30 minutes to about 48 hours at room temperature before coating.

The extrudate may be coated with one or more layers, although in some embodiments no coating is applied. The coating may be applied before segmenting into the desired insert length. The coating may be applied by dipping the extrudate into a liquid coating material and allowing it to dry or harden. This process may be repeated to add additional coating layers. Alternatively, the coating may be sprayed onto the extrudate.

In other embodiments, the coating/outer layer may be pre-formed in, e.g., a tube shape, and the API-polymer paste may be extruded into the tube.

Depending on the polymer used for the matrix, the matrix may be cured. Curing may be done, for example, by heating in an oven, microwave heating or chemical treatment. In other embodiments the matrix may not be cured. Instead it may be allowed to dry at air temperature or dried at a temperature of about 80° C. or lower.

In some embodiments, the matrix is uncured or is cured by heating at a temperature less than 80° C. In other embodiments the matrix is cured for about 10 minutes to about 300 minutes (5 hours) at a temperature of about 80° C. to about 160° C., about 15 minutes to about 4 hours at a temperature of about 80° C. to about 160° C., about 15 minutes to about 4 hours at about 120° C. to about 160° C., about 10 minutes to about 4 hours at about 130° C. to about 150° C., about 10 minutes to about 30 minutes at about 140° C. to about 160° C., about 30 minutes to about 4 hours at about 130° C. to about 150° C., about 200 minutes to about 1440 minutes at about 60° C. to about 120° C., about 300 minutes to about 600 minutes at about 60° C. to about 100° C., about 400 minutes to about 500 minutes at about 80° C. to about 90° C., about 600 minutes to about 1440 minutes at about 80° C. to about 120° C., about 800 minutes to about 1440 minutes at about 80° C. to about 110° C.

In additional embodiments, the matrix is cured for about 200 minutes to about 1600 minutes at about 90° C., about 200 minutes to about 500 minutes at about 90° C., about 500 minutes to about 1600 minutes at about 90° C., about 240 minutes at about 90° C., about 480 minutes at about 90° C., or about 1440 minutes at about 90° C.

In some embodiments, the matrix is cured for about 200 minutes to about 1600 minutes at about 100° C., about 200 minutes to about 500 minutes at about 100° C., about 500 minutes to about 1600 minutes at about 100° C., about 240 minutes at about 100° C., about 480 minutes at about 100° C., or about 1440 minutes at about 100° C.

In some embodiments, the matrix is cured for about 30 minutes to about 1600 minutes at about 110° C., about 30 minutes to about 200 minutes at about 110° C., about 200 minutes to about 1600 minutes at about 110° C., about 30 minutes at about 110° C., about 60 minutes at about 110° C., about 240 minutes at about 110° C. or about 1440 minutes at about 110° C.

In yet other embodiments, the matrix is cured for about 10 minutes to about 4 hours at about 140° C., about 10 minutes to about 1 hour at about 140° C., about 15 minutes to about 30 minutes at about 140° C., about 30 minutes to about 1 hour at about 140° C., about 1 hour to about 4 hours at about 140° C., about 1 hour to about 3 hours at about 140° C., about 10 minutes to about 400 minutes at about 140° C., about 30 minutes to about 400 minutes at about 140° C., about 60 minutes to about 380 minutes at about 140° C., about 60 minutes to about 300 minutes at about 140° C., about 180 minutes to about 300 minutes at about 140° C., about 220 minutes to about 280 minutes at about 140° C., about 230 minutes to about 300 minutes at about 140° C., or about 30 minutes to about 90 minutes at about 140° C.

Examples of curing temperatures include about 60° C. to about 100° C., about 60° C. to about 80° C., about 80° C. to about 100° C., about 80° C. to about 110° C., about 80° C. to about 120° C., about 85° C. to about 115° C., about 90° C. to about 100° C., about 90° C. to about 110° C., about 90° C. to about 120° C., about 90° C. to about 130° C., about 120° C. to about 140° C., about 130° C. to about 150° C., about 140° C. to about 160° C., about 135° C. to about 145° C., or about 140° C. to about 150° C.

Examples of curing times include about 20 minutes to about 400 minutes, about 30 minutes to about 400 minutes, about 60 minutes to about 400 minutes, about 90 minutes to about 400 minutes, about 120 minutes to about 400 minutes, about 180 minutes to about 360 minutes, about 200 minutes to about 320 minutes, about 200 minutes to about 300 minutes, about 20 minutes to about 240 minutes, about 20 minutes to about 200 minutes, about 20 minutes to about 180 minutes, about 20 minutes to about 120 minutes, about 20 minutes to about 90 minutes, about 20 minutes to about 60 minutes, about 30 minutes to about 120 minutes, and about 60 minutes to about 180 minutes.

In addition, examples of curing time include about 10 minutes, about 15 minutes, about 20 minutes, about 30 minutes, about 45 minutes, about 60 minutes, about 75 minutes, about 90 minutes, about 105 minutes, about 120 minutes, about 150 minutes, about 180 minutes, about 210 minutes, about 240 minutes, about 270 minutes, about 300 minutes, about 330 minutes, about 360 minutes, about 390 minutes, about 420 minutes, about 450 minutes, about 480 minutes, about 510 minutes, about 540 minutes, about 570 minutes, about 600 minutes, about 630 minutes, about 660 minutes, about 690 minutes, about 720 minutes, or about 1440 minutes. The curing temperature may be, for example, room temperature, about 60° C., about 65° C., about 70° C., about 75° C., about 80° C., about 85° C., about 90° C., about 95° C., about 100° C., about 105° C., about 110° C., about 120° C., about 125° C., about 130° C., about 135° C., about 140° C., about 145° C., about 150° C., about 155° C. or about 160° C.

After curing, the rods may be allowed to cool to room temperature before other manufacturing steps are performed. If the insert will be coated, the coating may be applied before or after curing.

Drug release rate was evaluated for both uncoated and PVA coated-PVA matrix inserts. The inventors found, generally, that the higher the curing temp and longer the curing period, the slower the drug release rate, but also the slower the erosion.

When all curing, cooling and/or coating and drying steps are complete, the rods are segmented into about 1 mm to about 15 mm long inserts, e.g., about 1 mm to about 10 mm, or about 2 mm to about 6 mm inserts. For example, the rods may be segmented into about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, or about 15 mm inserts.

The rods may be segmented, or otherwise cut into a series of shorter products, by any suitable technique for cutting the rods, which may vary according to whether the product is cured, uncured, or partially cured. For example, the segmenting station may employ pincers, shears, slicing blades, or any other technique. The technique applied may vary according to a configuration desired for each cut portion of the product. For example, where open ends are desired, a shearing action may be appropriate. However, where it is desired to seal each end as the cut is made, a pincer may be used.

In some embodiments the extrudates are dip coated in a solution of PVA in water with a concentration of PVA of about 1% w/w to about 20% w/w, about 1% w/w to about 15% w/w, about 1% w/w to about 10% w/w, about 2% w/w to about 10% w/w, about 2% w/w to about 8% w/w, about 2% w/w to about 6% w/w, about 3% w/w to about 6% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, or about 10% w/w.

The coated extrudates may then be air dried. The process of dip-coating may be repeated 1-10 more times, preferably 1-6 or 1-5 more times, and air dried between each coating. The coated extrudates may then be cured, as described above. After cooling, the extrudates are then cut into inserts.

e. Insert Properties

Some diseases of the eye, including those described herein, may require treatment for the remainder of the patient's life. Currently available therapies require repeated therapeutic treatments. However repetitive therapy by implantation of a drug delivery device into the eye is limited for devices that contain non-biodegradable materials, as the non-biodegradable remains of the devices accumulate in the eye. Thus, providing an implantable drug delivery device that fully erodes around the time, or shortly after, the next device needs to be implanted would be very beneficial to patients.

However, it is extremely challenging to design a drug delivery device that provides controlled release of therapeutics levels of a drug for a significant period of time that is also able to completely erode within, e.g., a matter of months or within about a year. Many materials that are effective at controlling drug release for significant periods are not bioerodible or erode too slowly.

In addition, providing a drug delivery device small enough to implant into the eye of a patient with minimal discomfort, yet that can contain a sufficient drug load to provide sustained delivery of the drug significantly increases the challenges described above. The difficulty of handling and processing such devices without significant breakage also adds to the challenges.

The inventors have overcome these challenges to provide a drug delivery device small enough to be implanted into the eye with minimal discomfort that is able to provide sustained delivery of the drug for months while also fully eroding sometime after the drug delivery period of the device has ended. In addition, the inventors have found a way to provide devices having different drug delivery periods/durations and rates of delivery. Moreover, these devices provide an essentially linear release of the drug after an initial burst of drug delivery. In addition, the insert has a very high drug content, relative to the inactive ingredients in the insert, which is surprising given the ability of the insert to provide release of the drug over extended periods.

i. Insert Erosion:

In some embodiments, the insert is capable of completely eroding within 365 days. The ability of an insert to erode within a given period of time may be evaluated using the following Erosion Evaluation Protocol. A sample insert is placed in a 10 mL glass vial with 5 mL phosphate buffered saline (PBS), the vial is incubated at 37° C., the PBS in the vial is replaced once every 24 hours for each day of the time period of interest (e.g., 365 days, 200 days, 110 days). At the end of this period, the insert is removed from the vial, allowed to dry, and then visually inspected and weighed. The reduction in weight as compared to the original weight is calculated as follows:

$$\frac{End Weight}{Start Weight} \times 100 = \% remaining$$

For example, if an insert that originally weighs 500 μg, and weighs 200 μg after incubating in PBS for 200 days according to the Erosion Evaluation Protocol, the insert weighs 40% of its original weight, and has lost 60% of its weight. It has undergone 60% erosion in 200 days. An insert is considered to be completely eroded when less than 10% of the original weight of the insert remains. In some embodiments, the insert completely erodes within 760 days, within 730 days, within 700 days, within 660 days, within 630 days, within 600 days, within 570 days, within 540 days, within 400 days, within 365 days, within 300 days, within 280 days, within 240 days, within 210 days, within 200 days, within 180 days, within 160 days, or within 140 days. In other embodiments, the insert is capable of at least 5% erosion within 60 days, at least 10% erosion within 60 days, at least 15% erosion within 60 days, at least 20% erosion within 60 days, at least 25% erosion within 60 days, at least 5% erosion within 75 days, at least 10% erosion within 75 days, at least 15% erosion within 75 days, at least 20% erosion within 75 days, at least 10% erosion within 95 days, at least 15% erosion within 95 days, at least 20% erosion within 95 days, at least 25% erosion within 95 days, at least 30% erosion within 95 days, at least 35% erosion within 95 days, at least 40% erosion within 95 days, at least 15% erosion within 100 days, at least 20% erosion within 100 days, at least 25% erosion within 100 days, at least 30% erosion within 100 days, at least 35% erosion within 100 days, at least 20% erosion within 110 days, at least 30% erosion within 110 days, at least 40% erosion within 110 days, at least 30% erosion within 180 days, at least 40% erosion within 180 days, at least 50% erosion within 180 days, at least 60% erosion within 180 days, at least 30% erosion within 220 days, at least 40% erosion within 220 days, at least 50% erosion within 220 days, at least 60% erosion within 220 days, at least 70% erosion within 220 days, at least 40% erosion within 280 days, at least 50% erosion within 280 days, at least 50% erosion within 280 days, at least 60% erosion within 280 days, at least 70% erosion within 280 days, at least 80% erosion within 280 days, at least 60% erosion within 365 days, at least 70% erosion within 365 days, at least 80% erosion within 365 days, at least 90% erosion within 365 days, at least 60% erosion within 400 days, at least 70% erosion within 400 days, at least 80% erosion within 400 days, at least 90% erosion within 400 days, at least 60% erosion within 440 days, at least 70% erosion within 440 days, at least 80% erosion within 440 days, or at least 90% erosion within 440 days measured using the Erosion Evaluation Protocol.

ii. Drug Release Rate:

The inventors found that curing temperature, curing duration and insert surface area all impact release rate. An increase in diameter with length kept constant, increased release rate. When the diameter was kept constant, increasing the length increased the release rate.

In some embodiments, the insert has a Drug Release Rate of about 0.01 μg/day to about 100 μg/day, about 0.01 μg/day to about 90 μg/day, about 0.01 μg/day to about 80 μg/day, about 0.01 μg/day to about 70 μg/day, about 0.01 μg/day to about 50 μg/day, about 0.01 μg/day to about 20 μg/day, about 0.01 μg/day to about 10 μg/day, about 0.1 μg/day to about 100 μg/day, about 0.1 μg/day to about 150 μg/day, about 0.1 μg/day to about 60 μg/day, about 0.1 μg/day to about 50 μg/day, about 0.1 μg/day to about 40 μg/day, about 0.1 μg/day to about 30 μg/day, about 0.1 μg/day to about 20 μg/day, about 0.1 μg/day to about 10 μg/day, about 0.1 μg/day to about 5 μg/day, about 0.1 μg/day to about 2 μg/day, about 0.1 μg/day to about 1 μg/day, about 0.5 μg/day to about 15 μg/day, about 0.5 μg/day to about 10 μg/day, about 0.5 μg/day to about 20 μg/day, about 0.5 μg/day to about 30 μg/day, about 1 μg/day to about 50 μg/day, about 1 μg/day to about 40 μg/day, about 1 μg/day to about 30 μg/day, about 1 μg/day to about 20 μg/day, about 1 μg/day to about 15 μg/day, about 1 μg/day to about 10 μg/day, about 5 μg/day to about 30 μg/day, about 5 μg/day to about 20 μg/day, or about 10 μg/day to about 30 μg/day. In some embodiments, this is the release rate after steady-state release is achieved. In some embodiments, this is the release rate after 2 days, 3 days, 5 days, 8 days, 10 days, 15 days, 20 days, 25 days, 30 days, 40 days, 50 days, 60 days, 70 days, 80 days, 90 days, 100 days, 105 days or 110 days of drug release. In some embodiments, the drug release rate is the average drug release rate, measured by the in vitro Drug Release Method (described below) over a specified period, e.g., 30 days, 60 days, 90 days, 120 days or 180 days. As used herein, the term “average release rate” refers to the sum total of the release rates of an ocular drug delivery insert over a period (e.g., 30 days) divided by the total number of days, to arrive at an average release rate. Average release rates are readily calculated by measuring the release rate for each day of the period using the methods described herein.

Thus, for example, in some embodiments the ocular drug delivery insert has an average drug release rate over a 30 day period of about 0.1 μg/day to about 150 μg/day.

In some embodiments, the insert has this release rate for at least 14 days, at least 30 days, at least 60 days, at least 90 days, at least 100 days, at least 120 days, at least 180 days, at least 200 days, at least 240 days, at least 270 days, at least 300 days, or at least 365 days, as measured by the in vitro Drug Release Method.

The following in vitro Drug Release Method is used to evaluate the amount of drug released: an insert is placed in a 10 mL glass tube, and 5 mL PBS is added to the tube. The tube is incubated in a water bath at 37° C. A sample of the medium is taken on each day of the stated period, and the release medium replaced with fresh PBS. The amount of API released may be measured quantitatively by HPLC as described in Example 2C.

The duration (total length of time) during which the insert releases API may be up to about 365 days, about 260 days, or about 200 days, or the duration may be at least about 8 weeks, at least about 10 weeks, at least about 12 weeks, at least about 18 weeks, at least about 22 weeks, at least about 28 weeks, at least about 30 weeks, at least about 36 weeks, at least about 40 weeks, at least about 44 weeks, or at least about 52 weeks. Alternatively, the duration of API release may be at least about 28 days, at least about 42 days, at least about 56 days, at least about 120 days, at least about 168 days, at least about 180 days, at least about 200 days, at least about 224 days, at least about 270 days, at least about 300 days, at least about 365 days, or at least about 730 days. The in vitro drug release method described above may be used to determine whether an insert releases drug for this duration.

In some embodiments, the insert of the invention provides an initial rapid release, or burst, of drug in vivo, for a period of time before achieving a steady state rate. In preferred embodiments of the invention, the initial period of rapid release is much less than total duration of API release (e.g., less than 10%). In some embodiments, this initial period is, e.g., 1 to 120 days, 20 to 120 days, 80 to 120 days, 1 to 20 days, 2 to 50 days, 3 to 40 days, 5 to 60 days, 1 day, 2 days, 3 days, 4 days, 5 days, 8 days, 10 days, 12 days, 15 days, 20 days, 25 days, 30 days, 40 days, 50 days, 60 days, 70 days, 80 days, 90 days, 100 days, 105 days, 110 days. In an in vitro study performed in rabbit eyes the inventors found a surprisingly high initial burst of drug release, meaning that the initial release of API from the insert was faster than expected, before leveling out to steady state. This burst may be beneficial as it allows Cₘₐₓ and equilibrium to be achieved quickly, thus providing therapeutically effective amounts locally to the eye quickly. After this burst the API release rate levels out to provide a therapeutically effective amount of the API each day.

In preferred embodiments, the insert of the invention releases the API at a substantially constant rate (i.e., zero-order drug release kinetics, R2 is from 0.7-1) over a predetermined duration after implantation. For example, it may release API at a substantially constant rate for about 14 days, about 28 days, about 42 days, about 56 days, about 168 days, about 180 days, about 224 days, about 270 days, about 300 days, or about 365 days. In some embodiments, the insert releases API at a substantially constant rate for at least 14 days, at least 28 days, at least 42 days, at least 56 days, at least 120 days, at least 168 days, at least 180 days, at least 224 days, at least 270 days at least 300 days, at least 365 days, at least 540 days, at least 600 days, or at least 730 days.

The duration of substantially constant API release from the insert may fall within a period of about 1 to about 48 months, about 2 to about 36 months, about 2 to about 24 months, about 2 to about 12 months, about 3 to about 9 months. In some aspects, the duration of substantially constant API release is about 60 days to about 730 days, about 60 days to about 540 days, about 60 days to about 365 days, about 60 days to about 300 days, about 60 days to about 270 days, about 90 days to about 365 days, about 90 days to about 270 days, about 180 days to about 365 days, or about 365 days to about 730 days. In some embodiments it is at least about 12 weeks, at least about 18 weeks, at least about 22 weeks, at least about 24 weeks, at least about 30 weeks, at least about 32 weeks, at least about 36 weeks, at least about 40 weeks, at least about 44 weeks, at least about 48 weeks, or at least about 52 weeks. The in vitro drug release test described above may be used to determine whether an insert releases drug for this duration.

### 3. Therapeutic Method

In some aspects, the insert is administered to inhibit VEGFR and/or PDGFR in an eye of a subject in need thereof. In other aspects, the insert is administered to inhibit angiogenesis in an eye of a subject in need thereof.

In other aspects, the ocular drug delivery insert is administered to prevent or treat a specific ocular condition or disease of the eye in a subject in need thereof, e.g., to treat an anterior ocular condition; to prevent an anterior ocular condition; to treat a posterior ocular condition; or to prevent a posterior ocular condition.

An “anterior ocular condition” is a disease, ailment, or condition that affects or involves an anterior (i.e., front of the eye, also referred to as the anterior segment) ocular region or structure, such as a periocular muscle or an eye lid, or a fluid located anterior to the posterior wall of the lens capsule or ciliary muscles. Thus, an anterior ocular condition can affect or involve the conjunctiva, the cornea, the anterior chamber, the iris, the posterior chamber (located between the iris and lens), the lens or the lens capsule and blood vessels and nerve which vascularize or innervate an anterior ocular region or site. An anterior ocular condition can include a disease, ailment or condition such as, but not limited to, glaucoma.

A “posterior ocular condition” is a disease, ailment or condition that primarily affects or involves a posterior (i.e., back of the eye, also referred to as the posterior segment) ocular region or structure, such as choroid or sclera (in a position posterior to a plane through the posterior wall of the lens capsule), vitreous, vitreous chamber, retina, optic nerve or optic disc, and blood vessels and nerves that vascularize or innervate a posterior ocular region or site.

A posterior ocular condition can include a disease, ailment or condition such as, but not limited to, acute macular neuroretinopathy; Behcet's disease; geographic atrophy; choroidal neovascularization; diabetic uveitis; histoplasmosis; infections, such as fungal, bacterial, or viral-caused infections; macular degeneration, such as neovascular macular degeneration, acute macular degeneration, age related macular degeneration (AMD) (such as non-exudative (dry) AMD, or exudative (wet) AMD (also known as advanced neovascular AMD)); edema, such as macular edema, cystoids macular edema, or diabetic macular edema (DME); multifocal choroiditis; ocular trauma which affects a posterior ocular site or location; ocular tumors; retinal disorders, such as retinal vein occlusion, central retinal vein occlusion, diabetic retinopathy (including proliferative and nonproliferative diabetic retinopathy), proliferative vitreoretinopathy (PVR), hypertensive retinopathy, retinal arterial occlusive disease such as central retinal artery occlusion (CRAO) and branch retinal artery occlusion (BRAO), retinal detachment, uveitic retinal disease; sympathetic ophthalmia; Vogt Koyanagi-Harada (VKH) syndrome; uveal diffusion; a posterior ocular condition caused by or influenced by an ocular laser treatment; or posterior ocular conditions caused by or influenced by a photodynamic therapy, photocoagulation, radiation retinotherapy, epiretinal membrane disorders, branch retinal vein occlusion, anterior ischemic optic neuropathy, non-retinopathy diabetic retinal dysfunction, and retinitis pigmentosa. Glaucoma may also be considered a posterior ocular condition because the therapeutic goal is to prevent the loss of or reduce the occurrence of loss of vision due to damage to or loss of retinal cells or optic nerve cells (e.g., via neuroprotection).

Thus, the invention provides methods of preventing or treating various ocular conditions by administering the ocular drug delivery insert to an eye of a subject in need thereof.

For example, the invention provides a method of treatment or prophylaxis of an ocular disease, by administering the ocular drug delivery insert to an eye in a subject in need thereof, wherein the ocular disease is characterized by damage to retinal neurons and/or to the optic nerve. In some embodiments, the disease may be geographic atrophy, glaucoma, diabetic macular edema, wet AMD, and retinal detachment. In some embodiments, the ocular disease is characterized by damage to photoreceptors.

In addition, the invention provides a method of providing neuroprotection to an ocular tissue by administering the ocular drug delivery insert to an eye in a subject in need thereof. For example, the invention provides a method of providing neuroprotection in the posterior segment of the eye, and, in particular, of providing neuroprotection in the retina. For example, the invention provides a method of providing neuroprotection to the retina to prevent diseases of the retina, such as dry AMD or wet AMD, or to slow the progression of diseases of the retina, e.g., to slow the progression of dry AMD to wet AMD, or slow progression through the stages of AMD.

In some embodiments, the ocular condition is diabetic macular edema (DME). In other embodiments, the ocular condition is retinal vein occlusion, such as central retinal vein occlusion (“CRVO”) or branch retinal vein occlusion (“BRVO”). In yet other embodiments, the ocular condition is non-ischemic retinal vein occlusion or ischemic retinal vein occlusion. In other embodiments the condition is diabetic retinopathy. In other embodiments, the condition is nonproliferative diabetic retinopathy.

In some embodiments, the inserts are administered to prevent or treat vision loss in a subject in need thereof, e.g., vision loss associated with macular degeneration.

In other embodiments of the methods of preventing or treating various ocular conditions by administering the ocular drug delivery insert to an eye of a subject in need thereof, the ocular condition is AMD.

The invention also provides a method of preventing the loss of visual acuity due to damage to or loss of retinal cells, such as retinal neurons. The invention further provides a method of reducing the occurrence of loss of vision due to damage to or loss of retinal cells, such as retinal neurons. In some embodiments of these methods, the administration of the ocular drug delivery insert reduces retinal thinning in the eye. In other embodiments, the administration of the ocular drug delivery insert protects against photoreceptor degeneration in the eye. The retinal cells may be, photoreceptors, bipolar cells, ganglion cells, horizontal cells, or amacrine cells.

### 4. Age Related Macular Degeneration

Age-related macular degeneration (AMD) is one of the most common causes of visual loss, projected to affect nearly 200 million people worldwide. Wong WL, Su X, Li X, et al. Global prevalence of age-related macular degeneration and disease burden projection for 2020 and 2040: a systematic review and meta-analysis. Lancet Glob. Health. 2014,2:e106-116.). Late-stage AMD, characterized by macular atrophy (known as geographic atrophy) and choroidal neovascularization (CNV), affects nearly 11 million people. Id. Roughly two thirds of the cases of late-stage AMD involve CNV, manifest by the exudation of fluid and blood, often resulting in vision loss and a fibrotic scar if untreated.

Age-related macular degeneration (AMD) can be divided into three stages, in part based on the number and size of drusen seen under the retina during a retinal examination. While dry AMD can fall into early, intermediate, and advanced stages, wet AMD is always considered an advanced form of AMD. Wet AMD includes Neovascular Age-Related Macular Degeneration, which is also an advanced form of AMD.

The following table describes four categories or stages of AMD, as defined in the National Eye Institute AREDS Study, according to the presence of certain markers of AMD. See https://www.nei.nih.gov/research/clinical-trials/age-related-eye-disease-studies-aredsareds2/about-areds-and-areds2.

Stages of Age-Related Macular Degeneration Defined in National Eye Institute AREDS Study

| Category | Description |
|---|---|
| 1 - No AMD | A few small (<63 μm in diameter) drusen or no drusen |
| 2 - Early-stage AMD | Many small drusen or a few medium-sized (63-125 μm in diameter) drusen in |
| | one or both eyes. |
| 3 - Intermediate AMD | Many medium-sized drusen or one or more large (≥125 μm in diameter) |
| | drusen in one or both eyes |
| 4 - Advanced AMD | In at least one eye, either a breakdown of light-sensitive cells and supporting |
| | tissue in the central retinal area (advanced dry form) or abnormal and fragile |
| | blood vessels under the retina (wet form) |

An individual may have AMD in one eye only (unilateral), or have AMD in both eyes (bilateral), but may be at different stages of AMD in each eye.

Where a subject has unilateral disease, or is at a more advanced stage of disease, the subject's other eye is referred to herein as the “fellow eye”. The fellow eye does not meet the diagnostic criteria for the disease with which the other eye has been diagnosed (e.g., wet AMD). For example, in a subject with unilateral wet AMD, there may be no evidence of choroidal neovascularization in the fellow eye.

Typically, there is no loss of vision in early AMD, and only small drusen or a few medium-sized drusen. Individuals with early AMD have a low risk of progressing to advanced AMD within 5 years.

Individuals with intermediate AMD are at significant risk for developing advanced AMD. In intermediate AMD, either multiple medium-sized drusen, or at least one large drusen, are found in one or both eyes, and changes in the retinal pigment epithelium (RPE), are also seen. The individual may have some or no vision loss in the affected eye(s).

There are two types of advanced AMD, dry (non-neovascular or non-exudative) AMD and wet (neovascular or exudative) AMD. An individual is more likely to progress to advanced AMD within 5 years, if the individual has large drusen in both eyes rather than in only one eye. An individual with advanced AMD is likely to have vision loss in the affected eye(s) because of macular damage. Having advanced AMD in one eye significantly increases the risk of developing advanced AMD in the other eye.

In dry AMD, photoreceptors (retinal cells sensitive to light) gradually die out, which impedes the eye's ability to sense light. This breakdown, along with degeneration of the retinal pigment epithelium (RPE), the underlying tissue that supports the retina, causes vision loss. The complete loss of RPE cells in an area is referred to a geographic atrophy.

In advanced wet AMD, abnormal blood vessels develop underneath the retina, referred to as choroidal neovascularization. These vessels may leak fluid or blood, thereby causing damage to surrounding tissue, including photoreceptors. Dry AMD may progress relatively slowly from early to advanced stages, or even not at all. However, wet AMD tends to progress quickly, and vision loss can occur suddenly from leakage or bleeding underneath or into the retina. It is also possible to have characteristics of both wet and dry AMD in the same eye.

Drusen deposits seen in intermediate or advanced dry AMD may enlarge and physically impinge on the photoreceptors and/or RPE. Larger drusen may lead to progressive tissue hypoxia and the release of factors such as VEGF and PLGF, which in turn stimulate increased vascular permeability, macular edema, exudation and choroidal neovascularization (CNV) seen in wet AMD. These new vessels are initially fragile, so they can break, leading to subretinal hemorrhage and photoreceptor toxicity. The progression of choroidal neovascularization can lead to disciform scar formation, also known as end-stage wet AMD. At this stage, treatment with drugs or surgery may provide no benefit.

Intravitreal injections of VEGF inhibitors can diminish the extent of exudation arising from CNV. Ranibizumab (Genentech, USA), bevacizumab (Genentech, USA) and aflibercept (Regeneron Pharmaceuticals, USA) are used worldwide to treat CNV secondary to AMD. However, these treatments have a risk of rare but serious adverse events resulting from the intravitreal procedure, and require monthly visits to a retinal specialist, which is a significant burden.

The invention provides methods of preventing or treating AMD by administering the ocular drug delivery insert to an eye of a subject. As described above, the ocular drug delivery insert of the invention can provide drug release for extended periods, which may result in fewer injections, fewer visits to a retinal specialist, and potentially better visual outcomes.

In certain embodiments, the insert is administered to prevent wet AMD in an eye of a subject. In other embodiments, the insert is administered to prevent choroidal neovascularization in an eye of a subject. In yet other embodiments, the insert is administered to prevent conversion of category 3 AMD to category 4 AMD in an eye of subject. In some embodiments, the insert is administered to slow the progression of AMD in an eye of a subject from an earlier stage to a later stage of AMD. In other embodiments, the insert is administered to stabilize the eye within a stage of AMD. In some embodiments the insert is administered to protect retinal pigment epithelial cells in the eye.

In certain embodiments of the method, the subject has unilateral wet AMD in the subject's other eye at baseline. In some embodiments, at baseline, the eye is in category 1 or has category 2 AMD. In other embodiments of the method, the subject has category 3 AMD in the subject's other eye at baseline. In yet other embodiments, the subject has category 3 AMD in both eyes at baseline. In some embodiments of the method, at baseline, the eye has category 3 AMD and the subject's other eye has category 4 AMD. In other embodiments, at baseline, the eye has category 2 AMD and the subject's other eye has category 4 AMD. In yet other embodiments of the method, at baseline, the eye is category 1 and the subject's other eye has category 4 AMD. In some embodiments of the method, an ocular drug delivery insert comprising vorolanib, or a pharmaceutically acceptable salt thereof, that releases about 0.1 μg/day to about 40 μg/day of vorolanib for at least 90 days and is capable of at least 20% erosion within 95 days, is administered to both of the subject's eyes.

Various methods may be used to diagnose AMD, to determine AMD stage, or to monitor the progression of an eye through the stages of AMD. Some of these diagnostic tools can detect choroidal neovascularization or changes in existing vascularization.

For example, an assessment of the best corrected visual acuity (BCVA) may be used to assess and monitor changes in vision as AMD progresses. In a subject with markers of AMD or at risk of AMD, changes in vision may be monitored by assessing BCVA at different timepoints. In addition, assessing BCVA before administering a particular AMD therapy and at various time points during therapy can help determine whether the therapy is effective at improving deleterious effects of AMD on vision, slowing AMD progression or stabilizing AMD. For example, an increase of BCVA of at least 5 ETDRS letters as compared to baseline, can indicate that a particular therapy is reducing the effects of AMD. Increases of, e.g., at least 10 ETDRS letters, or at least 15 ETDRS letters indicate a more significant improvement. Stabilization of vision, e.g., a loss of ≤15 ETDRS letters during treatment, in a subject with intermediate or advanced AMD, indicates that a therapy is stabilizing or slowing the progression of AMD. A loss of ≤10 ETDRS letters, or a loss of ≤5 ETDRS letters during treatment indicates a more significant effect on stabilization or slowing of AMD progression.

People with early stages of AMD may suffer from vision loss in low lighting, low contrast and/or changing light condition, which impacts their vision-related quality of life. The Impact of Vision Impairment (IVI) questionnaire, may be used to measure the impact of vision impairment on specific aspects of quality of life, has been found a reliable. See Weih, L. M., Hassell, J. B. & Keeffe, J. Assessment of the impact of vision impairment. Investigative ophthalmology & visual science 43, 927-935 (2002). The IVI has three vision-specific subscales: reading and accessing information, mobility and independence, and emotional well-being. A composite score is the total of the scores for all three subscales. In some embodiments of the invention, the IVI questionnaire composite score for the subject does not increase significantly from baseline for at least 180 days, at least 365 days or at least 545 days.

A dilated eye exam using an ophthalmoscope may be used to detect the presence of drusen in an eye and quantify and the number of drusen.

Fluorescein Angiography (FA) or Optical Coherence tomography (OCT) may be used to detect choroidal neovascularization, and to monitor neovascular changes and exudative changes in AMD, such as increase in lesion size. OCT may be either Spectral-Domain OCT (SD-OCT) or OCT-Angiography (OCT-A).

In some embodiments of the invention, no new choroidal neovascular lesions appear within 6 months from the date of administration, as compared to baseline. In other embodiments, existing choroidal neovascular lesions remain under 5 mm in diameter for at least 6 months after administration of the ocular drug delivery insert, as measured by OCT.

In some embodiments of the invention, administration of the ocular drug delivery insert prevents significant loss in visual acuity. For example, in some embodiments there is no change from baseline in BCVA of the eye to which the insert is administered for a certain period of time, where the period of time is measured from the day the ocular drug delivery insert is administered. In other embodiments, there is a loss of ≤5 ETDRS letters. In yet other embodiments, there is a loss of ≤10 ETDRS letters. In yet other embodiments there is a loss of ≤15 ETDRS letters. In some embodiments, there is a gain of ≥5 ETDRS letters. The period of time may be at least 90 days, at least 180 days, at least 270 days, or at least 365 days.

In some embodiments of the invention, administration of the ocular drug delivery insert prevents an increase in central subfield thickness (CST), also known as foveal thickness, (the average thickness of the macula in the central 1 mm ETDRS grid). For example, in some embodiments the CST of the eye to which the insert is administered does not increase over baseline for a certain period of time, where the period of time is measured from the day the ocular drug delivery insert is administered. In other embodiments, CST does not increase more than 100 μm, more than 75 μm, more than 50 μm, more than 25 μm, or more than 15 u μm during the period. In some embodiments of the invention, the IVI questionnaire composite score for the subject does not increase significantly from baseline for during the period.

In some embodiments of the method, there is no detectable choroidal neovascularization in the eye to which the insert is administered for a certain period of time, where the period of time is measured from the day the ocular drug delivery insert is administered. In other embodiments, the eye to which the insert was administered does not progress to an AMD category higher than the eye was at baseline for a certain period of time.

The period of time may be 90 days, 180 days, 270 days, 365 days, or 545 days. The particular test or evaluation method results at the end of the period are compared to baseline.

Herein, the eye at “baseline” can be evaluated just prior to administration of the ocular drug delivery insert, such as on day 0 (treatment day) or on one of the seven days prior to the day of administration (days −7 to −1).

In some embodiments of the invention, the eye is evaluated for evidence of AMD (e.g., drusen, BCVA, CST, or neovascularization) at baseline and then at one or more timepoints, such as at 30 days, 60 days, 90 days, 120 days, 150 days, 180 days, 210 days, 270 days, 300 days, 330 days, and/or 365 days after administration, with administration occurring on day 0. The eye may also be evaluated at additional timepoints.

The term “preventing”, when used in relation to a condition refers to administration of a drug to prevent the onset of or delay the onset of the ocular condition in a subject relative to a subject who does not receive the drug. The term “slow the progression of” a particular ocular condition means to prevent the worsening of that condition in a subject relative to a subject at the same stage of disease who does not receive the drug.

The term “treatment” means to diminish, ameliorate, or stabilize the existing unwanted condition.

a. Administering the Ocular Drug Delivery Insert

Administering the insert may comprise inserting the insert into an eye of a subject, such as inserting the insert into the aqueous humor or, preferably, into the vitreous humor of an eye. Administering the insert may comprise surgically implanting the insert into or onto an eye, such as a scleral implant, subconjunctival implant, suprachoroidal implant, suprascleral implant, or intravitreal implant. The insert can be surgically implanted into an eye of the subject, for example, into the vitreous of an eye, under the retina, or onto the sclera. In some embodiments, the insert may be placed by injection through a needle or cannula. The insert can gradually release an API in the eye, thus avoiding painful frequent administrations of the drug.

In certain embodiments, the insert is injected into an eye of the subject, preferably without requiring an incision. In certain aspects, the insert is injected into the vitreous of an eye. In preferred embodiments, administering the insert comprises intravitreal injection.

In some embodiments, a needle or cannula having a gauge size of 20-27 is used for the injection. In other embodiments, a needle or cannula having a gauge size of 25 to 27 is used. In preferred embodiments, a needle smaller than 25 gauge is used for the injection, e.g., a needle with a gauge of 25.5, 26, 26.5 or 27.

In some embodiments of the method of administration of the invention, before injection of the insert, a topical and/or subconjunctival anesthesia may be administered at the injection site. In addition, a broad-spectrum microbicide may be administered into the lower fornix. The insert may be place inferior to the optic disc and posterior to the equator of the eye. The conjunctiva may be displaced so that after withdrawing the needle, the conjunctival and scleral needle entry sites will not align. The needle used to inject the insert may be inserted through the conjunctiva and sclera up to the positive stop of the applicator, and the plunger depressed to deliver the insert into the back of the eye.

In some embodiments, an (one or more) insert is administered once every 90 days to 270 days, once every 90 days to 180 days, once every 120 to 720 days, once every 270 to 720 days, once every 270 to 540 days, once every 360 to 720 days, once every 360 to 540 days, or once every 540 to 720 days.

b. Subjects

In some embodiments, the ocular drug delivery insert is administered to an eye of a subject. In certain embodiments, the subject is a mammal. In further embodiments, the subject is a human.

c. Dose

In some embodiments, the total dose of vorolanib delivered is about 0.0001 μg/day to about 200 μg/day, about 0.0001 μg/day to about 150 μg/day, about 0.0001 μg/day to about 100 μg/day, about 0.0001 μg/day to about 80 μg/day, about 0.0001 μg/day to about 50 μg/day, about 0.0001 μg/day to about 30 μg/day, about 0.0001 μg/day to about 10 μg/day, about 0.0001 μg/day to about 5 μg/day, about 0.0001 μg/day to about 1 μg/day, about 0.001 μg/day to about 200 μg/day, about 0.001 μg/day to about 150 μg/day, about 0.001 μg/day to about 100 μg/day, about 0.001 μg/day to about 80 μg/day, about 0.001 μg/day to about 60 μg/day, about 0.001 μg/day to about 40 μg/day, about 0.001 μg/day to about 30 μg/day, about 1 μg/day to about 25 μg/day, about 0.001 μg/day to about 20 μg/day, about 0.001 μg/day to about 15 μg/day, about 0.001 μg/day to about 10 μg/day, about 0.001 μg/day to about 8 μg/day, about 0.005 μg/day to about 15 μg/day, about 0.005 μg/day to about 10 μg/day, about 0.01 μg/day to about 100 μg/day, about 0.01 μg/day to about 90 μg/day, about 0.01 μg/day to about 80 μg/day, about 0.01 μg/day to about 70 μg/day, 0.01 μg/day to about 50 μg/day, 0.01 μg/day to about 20 μg/day, 0.01 μg/day to about 10 μg/day, about 0.1 μg/day to about 150 μg/day, about 0.1 μg/day to about 100 μg/day, about 0.1 μg/day to about 80 μg/day, about 0.1 μg/day to about 60 μg/day, about 0.1 μg/day to about 50 μg/day, about 0.1 μg/day to about 40 μg/day, about 0.1 μg/day to about 30 μg/day, about 0.1 μg/day to about 20 μg/day, about 0.1 μg/day to about 10 μg/day, about 0.1 μg/day to about 5 μg/day, about 0.1 μg/day to about 2 μg/day, about 0.1 μg/day to about 1 μg/day, about 0.5 μg/day to about 15 μg/day, about 0.5 μg/day to about 10 μg/day, about 1 μg/day to about 50 μg/day, about 1 μg/day to about 40 μg/day, about 1 μg/day to about 30 μg/day, about 1 μg/day to about 20 μg/day, about 1 μg/day to about 15 μg/day, about 1 μg/day to about 10 μg/day, about 1 μg/day to about 5 μg/day, about 1 μg/day to about 10 μg/day, about 5 μg/day to about 30 μg/day, about 5 μg/day to about 20 μg/day, or about 10 μg/day to about 30 μg/day. In some embodiments, this is the release rate after steady-state release is achieved. In some embodiments, this is the release rate after 2 days, 3 days, 5 days, 8 days, 10 days, 15 days, 20 days, 25 days, 30 days, 40 days, 50 days, 60 days, 70 days, 80 days, 90 days, 100 days, 105 days or 110 days of drug release.

This dose may be achieved by administering, e.g., 1-6 inserts at one time, i.e., for a single treatment in one eye. Thus, one treatment may require administering 1 insert, 2 inserts, 3 inserts, 4 inserts, 5 inserts, or 6 inserts at one time per eye of a subject. In some embodiments a subject may receive treatment of only one eye, or of both eyes. Where more than one insert is injected for a single treatment, the inserts may be injected individually in separate injections, or a few inserts may be injected in one injection. For example, 1, 2 or 3 inserts may be injected in a single injection. Where more than 3 inserts are to be injected for a single treatment, they may be divided into a few injections. For example, if 4-6 inserts are to be injected for a single treatment, they may be divided to be administered in 2 or 3 injections of 2-3 inserts/injection.

Each insert may comprise about 1 μg to about 3000 μg, about 1 μg to about 1000 μg, about 1 μg to about 500 μg, about 10 μg to about 2000 μg, about 10 μg to about 1000 μg, about 100 μg to about 500 μg, about 10 μg to about 800 μg, about 50 μg to about 600 μg, about 200 μg to about 2000 μg, about 600 μg to about 2000 μg, about 800 μg to about 2000 μg, about 800 μg to about 1500 μg, about 100 μg to about 500 μg, about 100 μg to about 300 μg, or about 300 μg to about 550 μg of vorolanib. For example, each insert may comprise about 400 μg, about 420 μg, about 440 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1045 μg, about 1060 μg, about 1080 μg, or about 2000 μg of API, e.g., vorolanib.

The total amount of vorolanib in all of the inserts together (total payload) may be about 50 μg to about 1000 μg, about 200 μg to about 6000 μg, about 600 μg to about 6000 μg, about 800 μg to about 6000 μg, about 600 μg to about 5040 μg, about 600 μg to about 4500 μg, about 1000 μg to about 5400 μg, about 1000 μg to about 3000 μg, or about 2000 μg to about 4000 μg. For example, the total API amount for all inserts may be about 1400 μg, about 1420 μg, about 1500 μg, about 1600 μg, about 1800 μg, about 1900 μg, about 1980 μg, about 2000 μg, about 2040 μg, about 2080 μg, about 3000 μg, about 3120 μg, about 3180 μg, about 3240 μg, about 3400 μg, about 3600 μg, about 3800 μg, about 4000 μg, about 4140 μg, about 4160 μg, about 4180 μg, about 4200 μg, about 4400 μg, about 4600 μg, about 5000 μg, or about 5040 μg.

### 5. Combination—Induction Treatment and Maintenance Treatment

The invention also provides a method of treating a posterior ocular condition in an eye in need thereof, comprising, at a first timepoint, administering to the eye an agent that inhibits activation of VEGF receptors, such as a VEGF ligand, VEGF inhibitor or anti-VEGF (an induction treatment), and, at a second timepoint, administering to the eye an ocular drug delivery insert comprising vorolanib or a pharmaceutically acceptable salt thereof (a maintenance treatment) to maintain the induction treatment.

In some embodiments, the posterior ocular condition is selected from wet AMD, diabetic retinopathy (DR), macular edema following retinal vein occlusion (RVO), and diabetic macular edema (DME). In some embodiments, the posterior ocular condition is Neovascular Age-related Macular Degeneration

The first and second timepoints are suitably on different days. For example, the second time point may be at least about 1 week, at least about 2 weeks, at least about 4 weeks, at least about 8 weeks, or at least about 24 weeks, suitably at least 1 week, at least 2 week, at least 4 weeks, at least 8 weeks, or at least 24 weeks, after the first time point.

In general, the agent used for induction treatment (induction treatment agent) is any standard of care VEGF inhibitor (also sometimes referred to as an anti-VEGF). In some embodiments, the agent is a VEGF inhibitor selected from ranibizumab, bevacizumab, and aflibercept. In some embodiments, the VEGF inhibitor is administered by injection, e.g., by intravitreal injection. In some embodiments, the VEGF inhibitor is aflibercept injection for intravitreal use.

In some embodiments for treating wet AMD, the dose of aflibercept is about 2 mg or 2 mg (0.05 mL) administered by intravitreal injection every 4 weeks (approximately every 28 days, monthly). In some embodiments monthly injection administered for the first 12 weeks (3 months), followed by about 2 mg, or 2 mg (0.05 mL) intravitreal injection once every 8 weeks (2 months) or once every 12 weeks.

In some embodiments for treating for treating macular edema, the dose of aflibercept is about 2 mg or 2 mg (0.05 mL) administered by intravitreal injection once every 4 weeks (approximately every 25 days, monthly).

In some embodiments for treating for diabetic macular edema (DME) or diabetic retinopathy (DR), the dose of aflibercept is about 2 mg or 2 mg (0.05 mL) administered by intravitreal injection every 4 weeks (approximately every 28 days, monthly) for the first 5 injections followed by about 2 mg or 2 mg (0.05 mL) via intravitreal injection once every 8 weeks (2 months). In some embodiments, aflibercept is administered every 4 weeks (monthly) dosing after the first 20 weeks (5 months).

In some embodiments, the VEGF inhibitor is administered to the eye once per month, until the eye is dry or until no further visual or anatomical improvement is seen over baseline. The eye can then be assessed periodically, e.g., once every 2, 3, 4, 5, 6, 7 or 8 weeks. If fluid recurs, the VEGF inhibitor is administered again to the eye, and assessment continues. In some embodiments, the treatment interval for the VEGF inhibitor can be extended from once monthly (once every 4 weeks or 28 days), to once every 5 weeks or once every 6 weeks. If the eye remains fluid free for a given interval, the period over which the eye remains fluid free is assigned as the treatment interval for the VEGF inhibitor (e.g., treatment once every 2, 3, 4, 5, 6, 7, 8, 9, 10, 11, 12, 13, 14, 15, 16, 17, 18, 19 or 20 weeks). Assessment of the eye for fluid and/or visual acuity, will continue on a regular basis, e.g., once every 2, 3, 4, 5, 6, 7 or 8 weeks, and the treatment interval adjusted to be the same as the fluid free interval.

In some embodiments, the ocular drug delivery insert administered at the second timepoint (i.e., to an eye that has previously received an induction treatment) comprises a solid matrix core comprising a matrix polymer and vorolanib or a pharmaceutically acceptable salt thereof, wherein the amount of the vorolanib or pharmaceutically acceptable salt thereof in the insert is about 10% w/w to about 98% w/w, wherein the drug release rate for the insert is about 0.01 μg/day to about 100 μg/day for at least 14 days and wherein the insert is capable of at least 20% erosion within 95 days.

In further embodiments, the first dose of the ocular drug delivery insert is a loading dose, and later doses are maintenance doses, as described herein.

Thus, the invention provides a method that may be described as “a treat to maintain therapy” for posterior ocular conditions. In a majority of eyes, this treatment may result in a less intensive treatment regimen than treatment with a VEGF inhibitor alone and may keep the majority of eyes visually and anatomically stable for six months or longer. In some embodiments the eye is treated with one or more supplemental administrations of the VEGF inhibitor, after one or more doses of the ocular drug delivery insert have been administered. In some embodiments of the invention, the VEGF inhibitor is administered to an eye being treated concurrently with an ocular insert of the invention. Herein, concurrent treatment means that the eye to which a VEGF inhibitor is administered contains an ocular insert that is still releasing vorolanib.

In some embodiments, the VEGF inhibitor is administered before the first dose of the ocular drug delivery insert is administered as a loading dose, as described herein. In some embodiments, the first dose of the ocular drug delivery insert is administered to the eye that has been treated with VEGF inhibitor within about 1 week, at least about 2 weeks, at least about 4 weeks, at least about 8 weeks, at least about 12 weeks, or at least about 24 weeks.

In some embodiments, the first dose of the ocular drug delivery insert is administered to the eye that has previously responded to at least 2, 3, 4, 5, 6, 7, or 8 intravitreal injections of a VEGF inhibitor.

In some embodiments, the supplemental treatment is administered after administration of the ocular drug delivery insert loading dose described herein, e.g., while the loading dose ocular drug delivery insert is present in the eye.

In some embodiments, the supplemental treatment is administered after administration of the ocular drug delivery insert maintenance dose described herein, e.g., while the maintenance dose ocular drug delivery insert is present in the eye.

In some embodiments, aflibercept is administered as an induction treatment, as described herein, on day 1, on week 4, and on week 8. In some embodiments, the first dose of the ocular drug delivery insert is administered to the eye that has previously received an induction treatment on day 1, on week 4, and on week 8, 30 minutes after the induction treatment on week 8.

In some embodiments, starting from week 12, the supplemental treatment of aflibercept is administered to the eye that has previously received an induction treatment of aflibercept on day 1, on week 4, and on week 8 and received the first dose of the ocular drug delivery insert 30 minutes after the induction treatment on week 8. In some embodiments, the supplemental treatment of aflibercept is administered to the eye on week 12, wherein there is BCVA reduction of ≥5 letters from best on study measurement due to wet AMD and increase in CST of ≥75 microns on SD-OCT from lowest on study measurement, BCVA reduction of ≥10 letters from best on study measurement due to wet AMD, increase in CST of ≥100 microns on SD-OCT from lowest on study measurement from two consecutive visits, or presence of new or worsening vision-threatening hemorrhage due to wet AMD.

In some embodiments, the first ocular drug delivery insert is administered to an eye in which CST is less than 500 μm, 400 μm, 350 μm, 300 μm, 250 μm, or 200 μm. In some embodiments, the ocular drug delivery insert is administered to an eye in which CST is less than 500 μm, 400 μm, 350 μm, 300 μm, 250 μm, or 200 μm.

### 6. Definitions

As used in the specification and claims, the following words and phrases are generally intended to have the meanings as set forth below, except to the extent that the context in which they are used indicates otherwise.

The singular forms “a,” “an,” and “the” include plural reference unless the context clearly dictates otherwise. For example, “a matrix polymer” means one or more matrix polymers.

The terms “bioerode”, “bioerosion”, “biodegrade”, and “biodegradation” as used herein, refer to the gradual disintegration, dissolution, or breakdown of the insert over a period of time in a biological system, e.g., by one or more physical or chemical degradative processes, for example, enzymatic action, hydrolysis, ion exchange, or dissolution by solubilization, emulsion formation, or micelle formation.

The term “room temperature” means 22° C. “Solid at room temperature” means that solid at a temperature of 22° C.

When the term “about” is used in conjunction with a numerical value or range, it modifies that value or range by extending the boundaries above and below the numerical values set forth. In general, the term “about” is used herein to modify a numerical value above and below the stated value by a variance of 10 percent of the value, up or down (higher or lower), i.e., +10%, unless a different variance is indicated (e.g., +30%, +20%, +5%, +1%, +0.5% etc.).

The term “and/or” refers to and encompasses each of the listed items individually, as well as any and all possible combinations of one or more of the listed items.

The terms “comprising,” “consisting of” and “consisting essentially of” have their usual accepted meanings in accordance with patent law. When the term “includes” or “including” is used in the specification or the claims, it is intended to be inclusive in a manner similar to the term “comprising” as that term is interpreted when employed as a transitional word in a claim.

The terms “optional” and “optionally” mean that the subsequently described circumstance may or may not occur, so that the description includes instances where the circumstance occurs and instances where it does not.

Where features or aspects of the disclosure or claims are described in terms of Markush groups, the group described includes any individual member as well as subgroups of members of the Markush group.

As will be understood by one skilled in the art all language such as “up to,” “at least,” “greater than,” “less than,” include the number recited and refer to ranges which can be subsequently broken down into sub-ranges. Finally, as will be understood by one skilled in the art, a range includes each individual member, and includes the endpoints of the range. For example, a group having 1-3 members refers to groups having 1, 2, or 3 members. Similarly, a group having 1-5 members refers to groups having 1, 2, 3, 4, or 5 members, and so forth.

The term “substantially all” as used herein refers to most of the total amount, e.g., at least 80%, at least 85%, at least 90%, at least 95%, or at least 99% of a total amount.

The term % w/w means the proportion of a particular substance within a mixture, as measured by weight or mass. Thus, for example, for an insert in which the core comprises at least about 8% w/w of inactive ingredients, the total weight of inactive ingredients in the core is at least about 8% of the total weight of the core. For example, if the total core weight is 100 mg, the inactive ingredients in this core would weigh at least 8 mg.

The term % w/v means the percent of weight of ingredient (such as a solute) in the total volume of solution. A 2% w/v PVA solution would mean 2 grams of PVA in 100 mL of solution. A 2% w/w PVA solution would mean 2 grams of PVA for 100 mg of solution.

All cited patents, published applications, scientific publications and books are incorporated herein by reference in their entireties.

While various aspects and embodiments have been disclosed herein, other aspects and embodiments will be apparent to those skilled in the art.

### EXAMPLES

### Example 1

The grades of PVA listed in the table below were made into films, which were then evaluated for rate of erosion and strength.

To form films a 4.5% solution of PVA in water was cast by pouring into a tray and air drying at room temperature. When the films were dry, the films were cut into 1×1 square samples. Six samples of each film were then cured at 100° C. for 3 hours, 140° C. for 30 minutes or 140° C. for 4 hours, as described in the table below. After the sample film squares were cured, they were weighed and imaged. Then each sample was immersed in PBS for 24 hours at room temperature. The samples were then removed from the PBS, and 4 of each grade of PVA were oven dried at 50° C. for 2 hours, and 2 of each grade of PVA were air dried at room temperature on paper towels, as described in the table below. The samples were then weighed and imaged again.

TABLE 1

| | | | | Oven | Air |
|---|---|---|---|---|---|
| | 100° C./ | 140° C./ | 140° C./ | Dried | Dried |
| PVA MW/DH | 3 h | 30 min | 4 h | 50° C./2 h | at RT |
| 6000/80% | n = 6 | n = 6 | n = 6 | n = 4 | n = 2 |
| 25,000/88% | n = 6 | n = 6 | n = 6 | n = 4 | n = 2 |
| 78,000/98% | n = 6 | n = 6 | n = 6 | n = 4 | n = 2 |
| 125,000/88% | n = 6 | n = 6 | n = 6 | n = 4 | n = 2 |
| 1:9 Mixture of | n = 6 | n = 6 | n = 6 | n = 4 | n = 2 |
| 6000/80% and | | | | | |
| 78,000/98% | | | | | |
| 1:9 Mixture of | n = 6 | n = 6 | n = 6 | n = 4 | n = 2 |
| 6000/80% and | | | | | |
| 125,000/88% | | | | | |

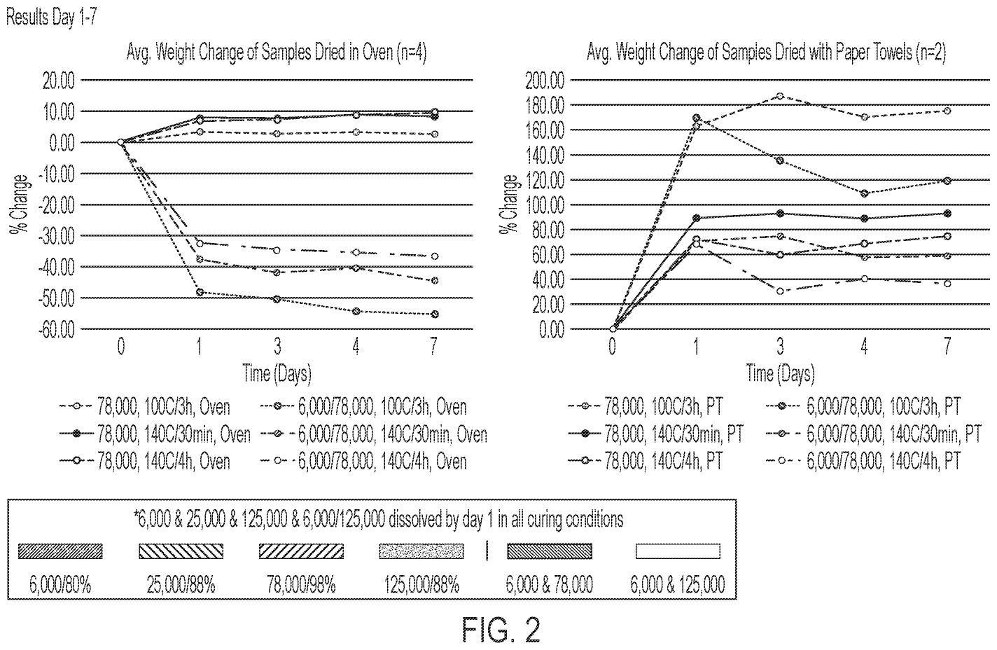

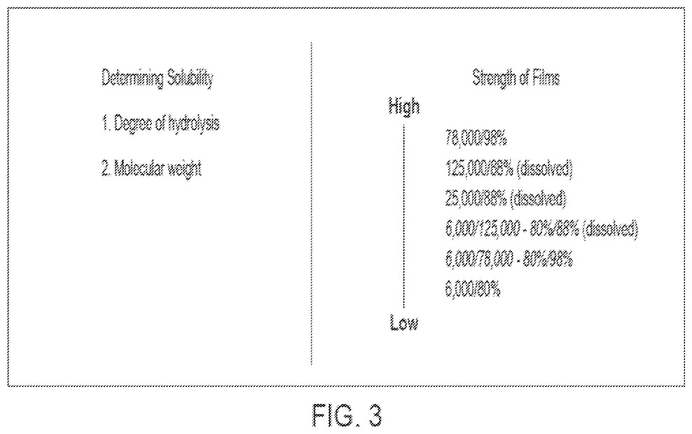

The weight average weight change after 24-hour immersion in PBS was calculated and is shown in graphs in FIG. 2. The PVA degree of hydrolysis (DH) and molecular weight (MW) determine the solubility of the films. The 6000/80%, 25,000/88%, 125,000/88%, and 6000/80%-125,000/88% mix PVA films each dissolved by the end of day 1 in all curing conditions tested. The 78,000/98% film lasted the longest. The relative film strengths of the films tested are depicted in FIG. 3.

### Example 2A

Inserts were made according to the parameters in the following table:

TABLE 2

| | | | | | | | | | | | |
|---|---|---|---|---|---|---|---|---|---|---|---|
| API:PVA | 1:1 | 1:1 | 1:1 | 1:1 | 1:1 | 1:1 | 1:1 | 1:1 | 1:1 | 1:1 | 1:1 |
| Ratio | | | | | | | | | | | |
| Core PVA | 4% | 4% | 4% | 4% | 4.5% | 4.5% | 4.5% | 4.5% | 4.5% | 4.5% | 5% |
| Soln. | | | | | | | | | | | |
| Coat PVA | 4% | 4% | 4% | 4% | 4.5% | 4.5% | 4.5% | 4.5% | 4.5% | 4.5% | 5% |
| Soln. | | | | | | | | | | | |
| No. Coats | 2, 4 | 4 | 1-4 | 1-4 | 4 | 4 | 2-4 | 2-4 | 2-4 | 0, 2-4 | 4 |
| Curing | 140° C. | 140° C. | 140° C. | 100° C. | none | 140° C. | 140° C. | 140° C. | 140° C. | 100° C. | 140° C. |
| | 1 h | 2 h | 4 h | 3 h | | 30 min | 1 h | 2 h | 3 h | 4 h | 4 h |

TABLE 3

| API:PVA | 1:1 | 1:1 | 1:1 | 1:1.35 | 1:1 | 1:1 | 1:1 | 1:1.35 | 1:1 | 1:1 | 1:1 | 1:1.35 |
|---|---|---|---|---|---|---|---|---|---|---|---|---|
| Ratio | w/w | w/w | w/w | w/w | w/w | w/w | w/w | w/w | w/w | w/w | w/w | w/w |
| Core | 4.5% | 6.5% | 9% | 9% | 4.5% | 6.5% | 9% | 9% | 4.5% | 6.5% | 9% | 9% |
| PVA | | | | | | | | | | | | |
| Soln. | | | | | | | | | | | | |
| No. Coats | No coating | | | | | | | | | | | |
| Curing | 140° C. | 140° C. | 140° C. | 140° C. | 140° C. | 140° C. | 140° C. | 140° C. | 140° C. | 140° C. | 140° C. | 140° C. |
| | 30 min | 30 min | 30 min | 30 min | 1 h | 1 h | 1 h | 1 h | 4 h | 4 h | 4 h | 4 h |

Inserts were manufactured by mixing vorolanib with a solution of 78,000/98% PVA in water in the w/w vorolanib:PVA solution ratio specified in the tables above. The mixture was then extruded from a 20, 21 or 23-gauge dispensing tip and dried at room temperature.

For inserts that were coated, the extrudate was then dip-coated in a 78,000/98% PVA solution and air dried. The process of dip-coating was repeated to achieve the number of coatings specified in the table above. The coating process involved dipping the extrudates in the PVA solution with 5 min room temperature drying between the first layers and then at least 10 min of drying time before dipping to form the last layer/coat. The coated extrudates were then cured as described in the table. After cooling to ambient temperature, the extrudates were cut into 2 mm, 3.5 mm, 5 mm or 6 mm or 8 mm long inserts.

### Example 2B

The drug release rate of the inserts was tested in vitro. Each insert sample was placed in a 10 mL glass tube, and 5 mL PBS is added to the tube. The tube was incubated in a water bath at 37° C. Samples of the release medium were taken at 12 to 24 hour intervals, and the release medium was replaced with fresh PBS. The amount of vorolanib released was measured quantitatively by HPLC according to the method described in Example 2C. The in vitro release rate was tested, and the average release rate was determined from the cumulative release versus time.

### Example 2C

Samples of the inserts were assayed for API content. The inserts tested for content were cut into 4 pieces and all 4 pieces were placed in a labeled scintillation vial. 3.0 mL of methanol was pipetted into the vial, and the vial was placed under a cabinet. This procedure was repeated for all samples. The sample vials were placed in a sonicator, an appropriate amount of water was added, and the samples were sonicated for 30 minutes. Sonication was repeated 5 more times, with the sonicator cooled between each sonication. Extra sonication can be done as needed to ensure the API is completely dissolved. HPLC was performed with the following parameters: Column: ZORBAX Eclipse XDB-C18; 4.6×150 mm; 5-micron; Mobile Phase A: Water+0.1% phosphoric acid; Mobile Phase B: Acetonitrile+0.1% phosphoric acid; Gradient Method; Stop time 30 min; UV: 214 nm.

### Example 2D

Erosion of sample inserts was evaluated. A sample insert was placed in a 10 mL glass vial with 5 mL phosphate buffered saline (PBS), and the vial was incubated at 37° C., without stirring. The PBS in the vial was replaced once every 24 hours for each day of the time period of interest. At the end of the period, the sample was removed from the vial, weighed and photographed.

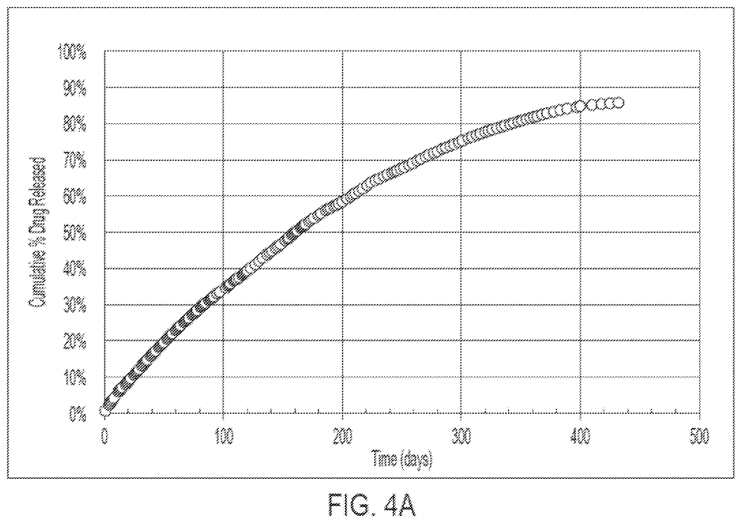

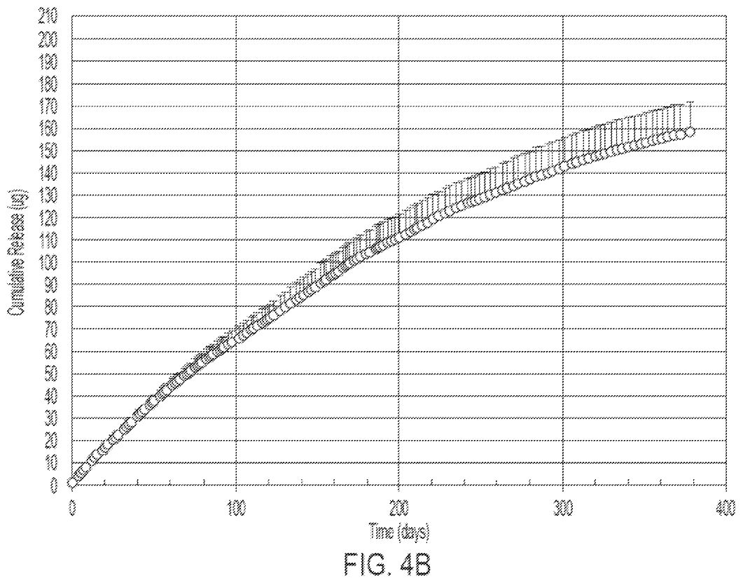

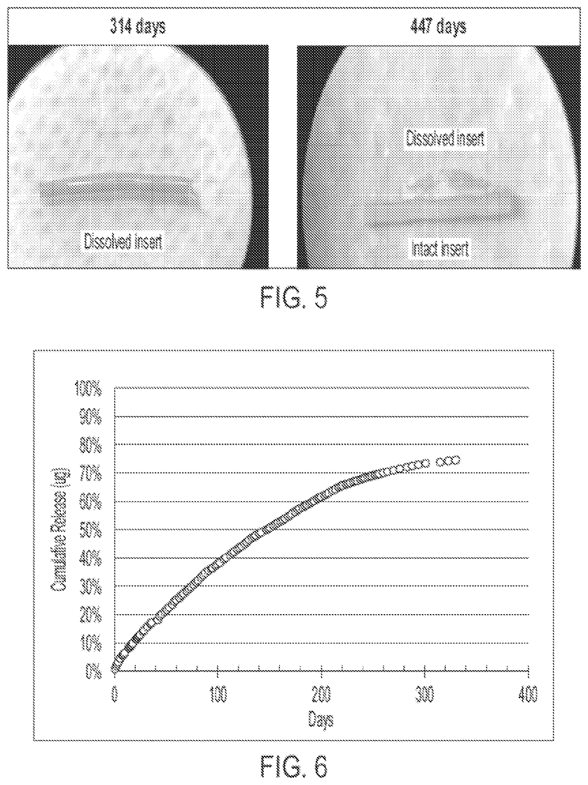

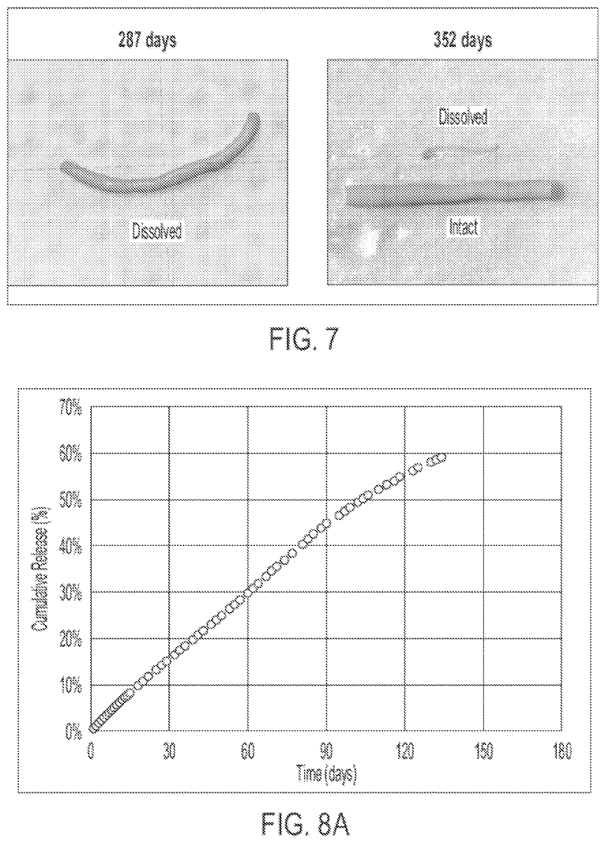

Drug release rate curves for a coated 4.5% PVA formulation cured at 140° C. for 4 hours, referred to as Formulation A, are shown in FIGS. 4A (cumulative % drug release) and 4B (cumulative drug release (μg)). The drug release rate curve for an Uncoated Formulation A insert is shown in FIG. 6. Photographs of eroded Formulation A inserts taken after immersion in dissolution medium for 314 and 447 days is shown in FIG. 5. An intact insert is included in the 447 day photograph for comparison. Photographs of eroded Uncoated Formulation A inserts taken after immersion in dissolution medium for 287 and 352 days is shown in FIG. 7. An intact insert is included in the 352 day photograph for comparison.

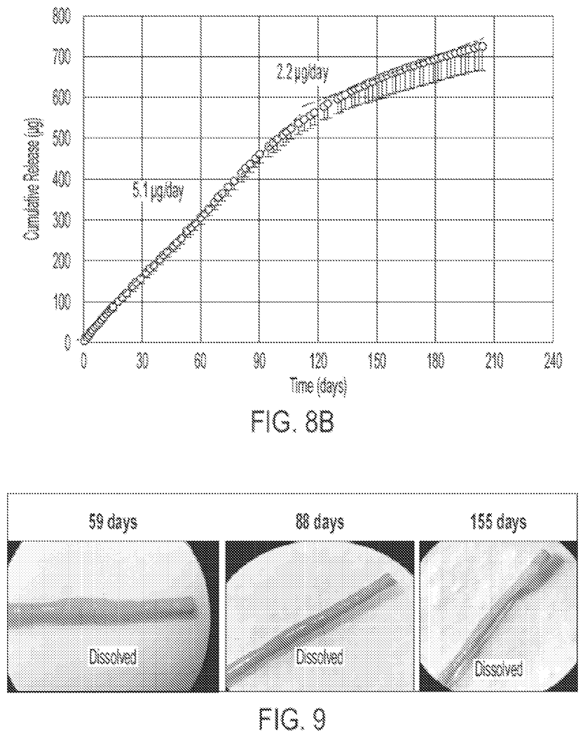

Drug release rate curves for a coated 4.5% PVA formulation cured at 140° C. for 30 minutes, referred to as Formulation B, are shown in FIGS. 8 (cumulative % drug release) and 8B (cumulative drug release (μg)). Photographs of eroded Formulation B inserts taken after immersion in dissolution medium for 59, 88 and 155 days are shown in FIG. 9.

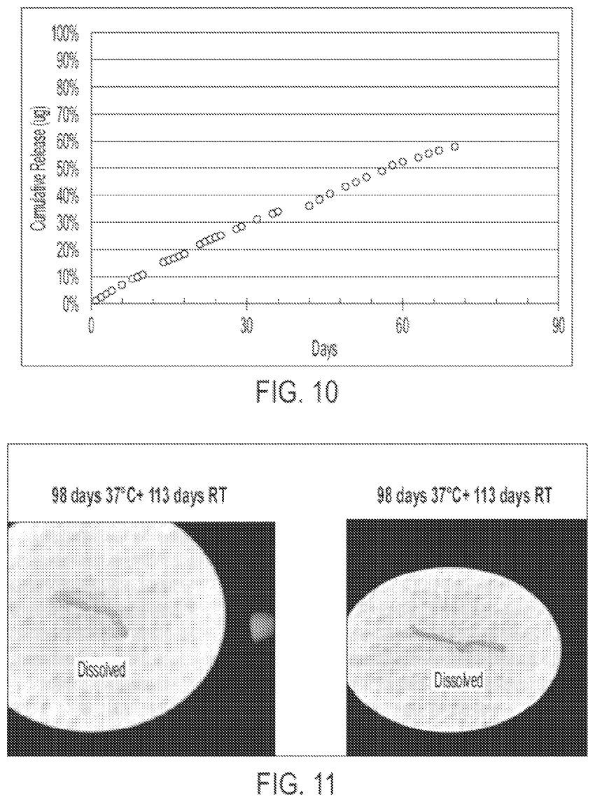

The drug release rate curve for an uncured coated 4.5% PVA formulation, referred to as Formulation C, is shown in FIG. 10. Two photographs each showing a different sample of an eroded Formulation C insert taken after immersion in dissolution medium for 98 days at 37° C. then 113 days at room temperature are shown in FIG. 11.

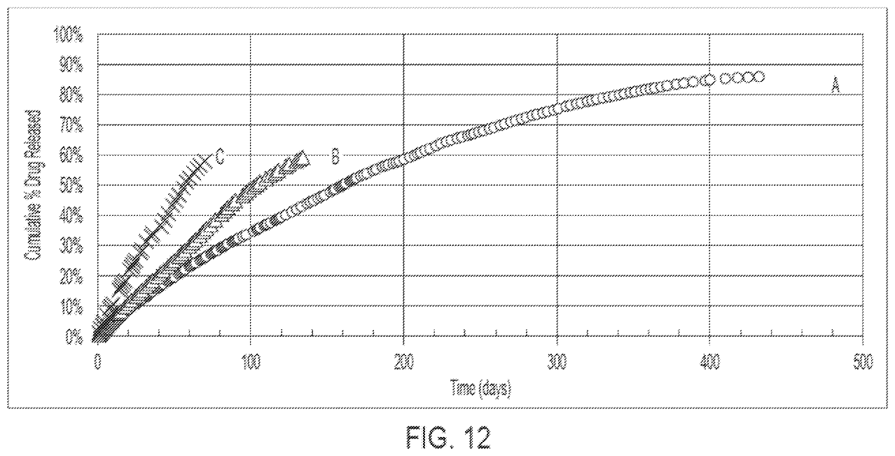

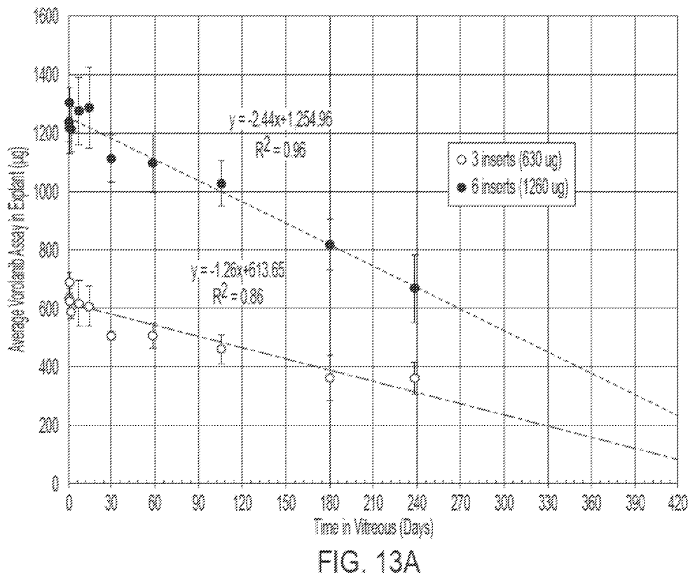

A comparison of the drug release curves for Formulations A, B and C is shown in FIG. 12.

Formulation A releases drug more slowly and erodes more slowly than Formulations B and C. Formulation C releases drug more quickly and erodes more quickly than Formulations A and B.

### Example 3

Inserts comprising more than one grade of PVA were made according to the parameters in the following table:

TABLE 4

| | | | | | |
|---|---|---|---|---|---|
| Core PVA | 4.5% | 4.5% | 4.5% | 4.5% solution | 4.5% solution |
| Solution | 78K/98% | 78K/98% | 78K/98% | mixture of 9:1 | mixture of 9:1 |
| | | | | 78K/98% and | 78K/98% and |
| | | | | 125K/88% | 125K/88% |
| Coat PVA | Coat: 4.5% | Coat 1: 4.5% | Coat: 4.5% | Coat: 4.5% | Coat: 4.5% |
| Solution | 78K/88% | 78K/98% | 125K/88% | 78K/98% | 125K/88% |
| | | Coats 2-4: 4.5% | | | |
| | | 78K/88% | | | |

| | |
|---|---|
| Curing | samples of each formulation were cured at 140° C./30 min |
| | samples of each formulation were cured at 140° C./1 h |
| | samples of each formulation were cured at 140° C./4 h |

Inserts were manufactured by mixing vorolanib with a solution of PVA in water in a 1:1 w/w vorolanib:PVA solution ratio to form a paste. The mixture was then extruded from a 21 gauge dispensing tip to form approximately 4-5 inch long rods and dried at room temperature. The extrudate rods were cured as described in the table above.

The extrudates were dip-coated in a solution of PVA in water and allowed to dry at room temperature. For inserts with more than one coat, the coating process involved dipping the extrudates in the PVA solution with 5 min room temperature drying between the first layers and then at least 10 min of drying time before dipping to form the last layer/coat.

After the last coat, the coated rods were cured according to the conditions described in the table above. After cooling to ambient temperature, the coated rods were cut into 8 mm long inserts using a razor blade.

API release rate was measured according to the method described in Example 2B.

API content was measured according to the method described in Example 2C.

Insert erosion was evaluated according to the method described in Example 2D.

Inserts comprising more than one grade of PVA are made according to the parameters in the following tables:

TABLE 5

| | | | | | |
|---|---|---|---|---|---|
| Core PVA | 4.0% | 4.5% | 4.0% | 4.0% solution | 4.0% solution |
| Solution | 78K/99+% | 78K/99+% | 89K-98K | mixture of 9:1 | mixture of 9:1 |
| | | | 99+% | 78K/98% and | 78K/98% and |
| | | | | 125K/88% | 125K/88% |
| Coat PVA | Coat: 4.5% | Coat: 4.5% | Coat: 4.5% | Coat: 4.5% | Coat: 4.5% |
| Solution | 78K/88% | 78K/88% | 78K/98% | 78K/98% | 125K/88% |

| | |
|---|---|
| Curing | samples of each formulation were cured at 140° C./30 min |
| | samples of each formulation were cured at 140° C./1 h |
| | samples of each formulation were cured at 140° C./4 h |

TABLE 6

| | | | | | |
|---|---|---|---|---|---|
| Core PVA | 4.5% | 4.5% | 4.5% | 4.5% solution | 4.5% solution |
| Solution | 78K/98% | 78K/99+% | 78K/88% | mixture of 9:1 | mixture of 9:1 |
| | | | | 78K/98% and | 78K/98% and |
| | | | | 125K/88% | 125K/88% |
| Coat PVA | Coat 1: 5.0% | Coats 1-3: 4.5% | Coats 1-5: 4.5% | Coats 1-2: 4.5% | Coat 1: 4.5% |
| Solution | 6000/80% | 78K/98% | 78K/98% | 78K/98% | 125K/88% |
| | | Coat 4: 5.0% | Coat 6: 5.0% | Coat 3: 4.5% | Coat 2: 4.5% |
| | | 6000/80% | 6000/80% | 78K/88% | 6000/80% |

| | |
|---|---|
| Curing | samples of each formulation were cured at 140° C./30 min |
| | samples of each formulation were cured at 140° C./1h |
| | samples of each formulation were cured at 140° C./4h |

Inserts are manufactured by mixing vorolanib with a solution of PVA in water in a 1:1 w/w vorolanib:PVA solution ratio to form a paste. The mixture is then extruded from a 21 gauge dispensing tip to form approximately 4-5 inch long rods and dried at room temperature. The extrudate rods are cured as described in the tables above.

The extrudates are dip-coated in a solution of PVA in water and allowed to dry at room temperature. For inserts with more than one coat, the coating process involves dipping the extrudates in the PVA solution with 5 min room temperature drying between the first layers and then at least 10 min of drying time before dipping to form the last layer/coat.

After the last coat, the coated rods are cured according to the conditions described in the table above. After cooling to ambient temperature, the coated rods are cut into 8 mm long inserts using a razor blade.

API release rate is measured according to the method described in Example 2B.

API content is measured according to the method described in Example 2C.

Insert erosion is evaluated according to the method described in Example 2D.

### Example 4

Inserts are made according to the parameters in the following tables:

TABLE 7

| API | Vorolanib | | | | | | | | | |
|---|---|---|---|---|---|---|---|---|---|---|
| API:PVA | 1:1 w/w | | | | | | | | | |
| Core | 2.5% | 2.5% | 2.5% | 2.5% | 3% | 3% | 3% | 3% | 5% | 5% |
| PVA | | | | | | | | | | |
| Solution | | | | | | | | | | |
| Coat | 4.5% | 4.5% | 5% | 5% | 5% | 5% | 5% | 5% | 5% | 5% |
| PVA | | | | | | | | | | |
| Solution | | | | | | | | | | |
| No. Coats | 2-4 | 2-4 | 1-4 | 1-4 | 0-4 | 0-4 | 0-4 | 0-4 | 1-4 | 0-4 |
| Curing | after | after | after | before | after | after | after | after | after | after |
| | coating | coating | coating | coating | coating | coating | coating | coating | coating | coating |
| | 140° C. | 140° C. | 140° C. | 140° C. | 150° C. | 140° C. | 140° C. | 140° C. | 150° C. | 140° C. |
| | 2 h | 4 h | 4 h | 4 h | 30 min | 2 h | 3 h | 4 h | 30 min | 2 h |

TABLE 8

| API | Vorolanib | | | | | | | | | |
|---|---|---|---|---|---|---|---|---|---|---|
| API:PVA | 1:1 w/w | | | | | | | | | |
| Core | 5% | 5% | 4% | 4% | 4% | 5% | 5% | 8% | 10% | 12% |
| PVA | | | | | | | | | | |
| Solution | | | | | | | | | | |
| Coat | 6% | 6% | 8% | 8% | 10% | 8% | 8% | 4.5% | 4% | 4% |
| PVA | | | | | | | | | | |
| Solution | | | | | | | | | | |
| No. Coats | 1-4 | 1-4 | 1-4 | 1-4 | 1-4 | 1-4 | 1-4 | 0-4 | 0-4 | 0-4 |
| Curing | after | after | after | before | after | after | before | after | after | after |
| | coating | coating | coating | coating | coating | coating | coating | coating | coating | coating |
| | 140° C. | 140° C. | 140° C. | 140° C. | 140° C. | 140° C. | 140° C. | 140° C. | 140° C. | 140° C. |
| | 2 h | 4 h | 4 h | 4 h | 30 min | 2 h | 3 h | 2 h | 30 min | 2 h |

The API is mixed with a solution of PVA in water in the API:PVA solution ratio specified in the table to form a paste. The paste is extruded through a dispensing tip with a gauge of 20-23 to form approximately 4-5 inch long rods and dried at room temperature. The extrudate rods are cured before or after coating as described in the tables above.

The extrudates are dip-coated in a solution of PVA in water. The coating process involves dipping the extrudates in the PVA solution with 5 min room temperature drying between the first layers and then at least 10 min of drying time before dipping to form the last layer/coat. After the last coat, the coated rods either cured according to the tables above or allowed to dry for 24 hours at room temperature.

The coated rods are cut into 2 mm, 3.5 mm, 5 mm or 6 mm long inserts using a razor blade.

API release rate is measured according to the method described in Example 2B.

API content is measured according to the method described in Example 2C.

Insert erosion is evaluated according to the method described in Example 2D.

### Example 5A—Pharmacokinetics Study

An Intravitreal Pharmacokinetic Study was performed in Male Dutch Belted Rabbits. The objective of this study was to characterize the plasma and ocular tissue pharmacokinetics of a vorolanib insert following bilateral intravitreal injection on Day 1. Animals were evaluated up to 24 months following placement of the intravitreal insert.

The table below describes the group assignment dose levels and treatments.

TABLE 9

Treatment

| Group | OD (Rt. Eye) | OS (Lft. Eye) |
|---|---|---|
| Group 1 Low Dose | 3 inserts | 3 inserts |
| Group 2 High Dose | 6 inserts | 6 inserts |

After administration of anesthesia, vorolanib inserts measuring 0.37 mm in diameter by 3.5 mm in length designed to release drug for at least 6 months were injected, using an injector, intravitreally into each eye of 52 male Dutch-belted rabbits. The low dose group (1) received 3 inserts per eye for a total dose of 630 μg per eye. The high dose group (2) animals received 6 inserts per eye given in 2 separate injections (3 inserts per injection) for a total dose of 1,260 μg per eye.

Prior to each scheduled sacrifice point, one whole blood sample was collected from a targeted 2 animals per group via puncture of a marginal ear vein. Samples were tested for levels of vorolanib and its metabolite. Two animals per group were euthanized on day 1 at 6, 12, 24 and 48 hours, on days 7 and 14, and at 1, 2, 4, 6, 8, 16 and 24 months. Vitreous humor and ocular tissues were collected from both eyes for analysis of ocular tissue drug distribution, inserts were collected, and samples of liver and kidney were collected for evaluation of tissue distribution.

Results: At steady state, the vitreous level of vorolanib was 56 ng/ml for the low dose of 630 μg, and 97 ng/mL for the high dose of 1,260 μg (˜dose proportionality). Retina/choroid levels were 49 ng/g and 89 ng/g. There was a burst period of drug release in the first 90 days, followed by steady state. Steady state was reached by day 105. The maximum observed concentrations in vitreous and retina/choroid appear to be nearly dose proportional. No apparent change was found in plasma levels after 99 days. Through day 180, the vitreous Cₘₐₓ was 232 ng/ml, Tₘₐₓ was 336 h, and AUCₗₐₛₜ was 315.5 μg·h/mL for the 630 μg dose. Through day 180, the vitreous Cₘₐₓ was 1697 ng/ml, Tₘₐₓ was 720 h, and AUCₗₐₛₜ was 1583.2 μg·h/mL for the 1260 μg dose.

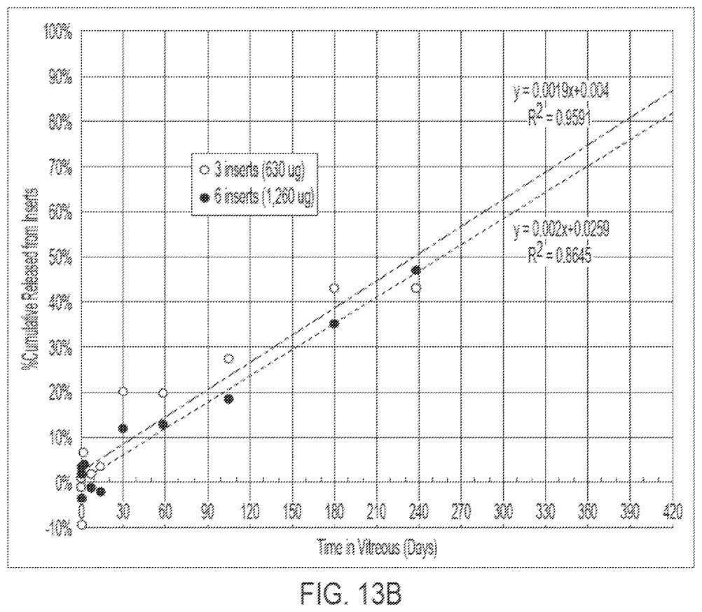

FIG. 13A depicts average amount of drug remaining in an insert versus time for inserts explanted at various time points and assayed to determine the amount of vorolanib remaining in the insert. FIG. 13B depicts cumulative percent of drug released versus time for explanted inserts.

### Example 5B—Pharmacokinetics Study

A second Intravitreal Pharmacokinetic Study was performed in Dutch Belted Rabbits. The objective of this study was to evaluate the plasma and ocular pharmacokinetics of an vorolanib insert following bilateral intravitreal injection in rabbit eyes. Animals were evaluated up to 12 months following placement of the intravitreal insert.

Dutch Belted rabbits were administered vorolanib inserts in each eye by intravitreal injection. Group 1 eyes received 1 insert containing 643 μg of vorolanib, and Group 2 eyes received 2 inserts containing 900 μg total. Blood samples were collected at 2, 7, 14, and 28 days and then once monthly for months 2-12. Inserts were recovered at 2, 7, and 14 days and then at 1, 2, 4, 6, 8, 10, and 12 months. Residual vorolanib levels were determined in all explants using high-performance liquid chromatography. The vorolanib release rate was estimated based on residual levels in the explants. Plasma and ocular tissues were separated and analyzed for vorolanib and its metabolite using liquid chromatography mass spectrometry.

Results: The inserts released vorolanib through 12 months at an average rate of 8.1%/month in Group 1 and 7.8%/month in Group 2. The release profile displayed near zero-order kinetics through 8 months, demonstrating consistent release of microgram levels of drug each day, with concentrations in target ocular tissues (choroid and retina) above the IC50 for VEGFR2. Beyond 8 months the release rate dropped rapidly, yielding target ocular tissue levels below IC50 (<10 ng/ml) by 10 months. Vorolanib exposure rank order was choroid=retina=vitreous>aqueous humor>plasma. There was an 85%-99% drop in mean vorolanib concentration in ocular tissues and plasma between months 8 and 10.

The vorolanib insert demonstrated sustained and consistent zero-order release of vorolanib in rabbit eyes through 8 months followed by a rapid decrease through 10 months. A vorolanib insert is being studied in phase 2 clinical trials in wAMD and diabetic retinopathy, and a trial in diabetic macular edema is planned.

### Example 6—Toxicology and Pharmacokinetics Study

An 18 month intravitreal toxicity study for the insert was also performed in 80 Dutch Belted rabbits (40 male and 40 female). Animals were evaluated for a period of 6 and 18 months following placement of intravitreal inserts. The objective was to characterize the ocular toxicity, plasma pharmacokinetics and biodegradation of a vorolanib insert following bilateral intravitreal injection.

The tables below describe the group assignments and dose levels. For the toxicology groups, animals were sacrificed at 6 months and 18 months. For the plasma pharmacokinetic analysis, blood samples were collected at Days 1, 3, and 7 and then 1, 2, 3, 4, 5, 6, 12, 14, 16 and 18 Months.

TABLE 10

Treatment

| Group | OD (Rt. Eye) | OS (Lft. Eye) |
|---|---|---|
| Group 1 Control | 2 placebo inserts | 2 placebo inserts |
| Group 2 Low Dose | 2 inserts | 2 inserts |
| Group 3 Mid Dose | 3 inserts | 3 inserts |
| Group 4 High Dose | 4 inserts | 4 inserts |
| Group 5 Highest Dose | 6 inserts | 6 inserts |

After administration of anesthesia, vorolanib inserts measuring 0.37 mm in diameter by 3.5 mm in length designed to release drug for at least 6 months were injected, using an injector, intravitreally into each eye of each Dutch-belted rabbit. The placebo group (1) animals received two placebo inserts by injection in each eye. The low dose group (2) animals received 2 inserts in each eye. The mid dose group (3) animals received 3 inserts in each eye given in 2 separate injections. The high dose group (4) animals received 4 inserts in each eye given in 2 separate injections (2 inserts/injection). The highest dose group (5) animals received 6 inserts in each eye given in 2 separate injections (3 inserts/injection).

At each scheduled timepoint, whole blood was collected via puncture of a marginal ear vein. Samples were analyzed for clinical pathology and plasma pharmacokinetics. Animals were euthanized according to the schedules described above. A complete gross necropsy was performed on all animals that were sacrificed or found dead during the study. Organs were weighed and tissues collected. Ocular tissues were collected for histopathology only.

Conclusions: The plasma pharmacokinetic and toxicology studies provide evidence of safety at vitreous Cₘₐₓ and AUC over an 18 and 24 month period of exposure, respectively. In addition, at the time points tested, vorolanib levels in the vitreous and the retina/choroid remained significantly above the IC₅₀ for VEGFR.

No adverse findings were attributed to vorolanib and there were no adverse findings for up to 6 inserts. The no observed adverse effect level (NOAEL) for the inserts was determined to be 6 inserts/eye (1260 μg/eye).

The highest observed event is yellow discoloration of the lens, which appears to be dose related and due to API color. There were no histopathological/microscopic findings associated with lens discoloration. The second highest observed event is focal, punctuate or linear lens opacity and appears to be related mostly to the number of injections and, to a lesser degree, the number of inserts.

Mild inflammation (<2+ aqueous or vitreous cells) was observed across all groups initially. All inflammatory cells had gradually resolved and cleared by 3 months. The highest observed events for inflammation were seen in the placebo group (2 inserts w no drug).

There was no change in intraocular pressure (IOP) from baseline, although some transient changes were observed.

vorolanib plasma levels were in the low pg/mL range.

### Example 7 Safety and Efficacy

Safety and efficacy of a vorolanib insert were evaluated in a Swine (Mini-pig) Model of Laser-Induced Choroidal Neovascularization (CNV). The primary objective of this study was to evaluate the long-term safety and inhibition of vascular permeability and neovascularization in a laser-induced model of choroidal neovascularization (CNV) using a vorolanib insert in swine.

The experimental design is described in the following table:

TABLE 11

| | Test Article | Volume/ | Admin. | CNF Laser/ | | |
|---|---|---|---|---|---|---|
| Group | & Dose | Route | Day | Dosing Day | Experimental Endpoint | Euthanasia |
| 1 | Aflibercept | 50 μL/ | 0 | OU: CNV | OEs: Baseline, prior to | Day 28 |
| | (2 mg) | eye IVT | | laser (D 0) | laser, and Days 7, 14, | |
| 2 | Low dose | 1 insert/ | −7 | | and 28. | |
| | | eye IVT | | | Fluorescein Angiography: | |
| 3 | High dose | 2 inserts/ | −7 | | Day 7, 14, and 28. | |
| | | eye IVT | | | Histology: Collect eyes | |
| 4 | Placebo | 2 inserts/ | −7 | | for potential histological | |
| | | eye IVT | | | examination | |
| 5 | High dose | 2 inserts/ | 0 | None | OEs: Baseline, prior to | Day 84 |
| | | eye IVT | | | laser, and Days 7, 14, 28, | |
| | | | | | 56 and 84. | |
| | | | | | ERGs: Baseline, prior to | |
| 6 | Placebo | 2 inserts/ | 0 | | necropsy | |
| | | eye IVT | | | Histology: Collect eyes | |
| | | | | | for histological | |
| | | | | | examination | |

On the day of lasering (Groups 1-4) animals were treated with an 810 nm diode laser delivered through an indirect ophthalmoscope. Approximately 6 single laser spots were placed between retinal veins. Both eyes underwent laser treatment according to the schedule in the table above.

On the day of intravitreal injections animals were anesthetized and the eyes were aseptically prepared. The conjunctiva was gently grasped with colibri forceps, and the injection (25G injector needle) was made 2-3 mm posterior to the superior limbus (through the pars plana), with the needle directly slightly posteriorly to avoid contact with the lens. Animals were allowed to recover normally from the procedure. The pigs were given a topical drop of antibiotic ophthalmic solution 4-6 hours later, and then BID for 2 additional days with at least 6 hours between doses. Animals were dosed on either Day 0 immediately following laser CNV induction or 7 days prior to laser CNV induction according to the schedule in the table above. Animals in Groups 5-6 did not undergo laser CNV procedures and were implanted on Day 0.

During acclimation, and while on study, the animals were evaluated for mortality and morbidity as well as general health, with particular attention to the eyes. Body weights were taken prior to treatment and prior to necropsy, animals in Groups 5 & 6 were weighed monthly.

Complete ocular examination (OE) (modified Hackett and McDonald) using a slit lamp biomicroscope and indirect ophthalmoscope to evaluate ocular surface morphology, anterior segment and posterior segment inflammation, cataract formation, and retinal changes was conducted by a veterinary ophthalmologist at the timepoints as indicated in the experimental design table. Mydriasis for ocular examination was done using topical 1% tropicamide HCL.

Flourescein Angiography was done in both eyes in anesthetized animals at the timepoints as indicated in the experimental design table.

Full-field electroretinography (ERG) was done on both eyes of the animals at baseline and 3 months following dosing (Groups 5-6 only). On the day of ERG measurements, animals were anesthetized following dark adaptation. ERGs were elicited by brief flashes at 0.33 Hz delivered with a mini-ganzfeld photostimulator at maximal intensity. Twenty responses were amplified, filtered, and averaged for each animal. Animals underwent standard ERG measurements as dictated by ISCEV standards, including scotopic (0.01 candela), scotopic (3 candela), and photopic (25 candela) measurements.

At time points indicated by the experimental design table, following final data collections, animals were euthanized. Histology was evaluated for eyes from Groups 5-6.

Results: Overall, dose-related efficacy was found and there was no clinically observed toxicity. Fluorescein angiography analysis in all groups (1-4) had a reduced Corrected Total Lesion Fluorescence (CTLF) value from Day 7 to Day 28, with aflibercept treated animals having the largest reduction in CTLF values, followed by high dose, and the remaining groups having similar reductions in CTLF values. ERG b-wave amplitudes were reduced from baseline to Day 84 in both groups undergoing ERGs; however, this may be attributed to difficulties in ERG acquisition.

Eyes undergoing histological examinations displayed some inflammation, which may have been more severe in animals dosed with high dose inserts. The implant procedure may have contributed to the increased inflammation observed in the high dose group.

Aflibercept and placebo inserts performed as expected, with aflibercept having normal amounts of efficacy in this model, and placebo inserts being well tolerated.

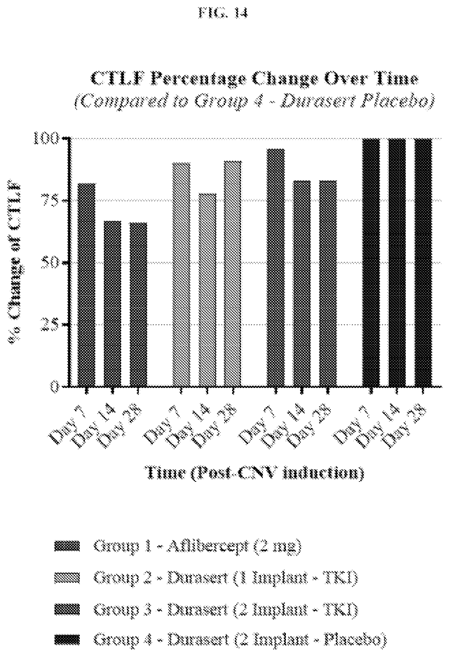

As can be seen in FIG. 14, the lesions in the Group 2 (medium dose) and Group 3 (high dose) animals, which received inserts 7 days before laser CNV, never reached the size of the lesions in untreated eyes. This indicates the inserts had prevented lesions from reaching the size seen in the untreated eyes. FIG. 14 is a bar graph comparing the Corrected Total Lesion Fluorescence (CTFL) Percentage Change over time for Groups 1-4.

### Conclusions

Vorolanib plasma levels in the PK study were in the low pg/mL range. Dose-related efficacy was found and there was no clinically observed toxicity. Thus, the inserts of the invention were able to deliver safe and therapeutically effective steady state levels of vorolanib locally over a sustained period, while resulting in only negligible systemic levels of vorolanib. Moreover, the inserts are fully bioerodible. In addition, the inserts appear to have a preventative effect on lesion growth.

### Example 8: DAVIO, a Phase 1, Multicenter, Prospective, Open-Label, Dose Escalation Study of EYP-1901, a Tyrosine Kinase Inhibitor (TKI) Ocular Drug Delivery Insert, in Subjects with Wet AMD

A phase 1, open-label, dose-escalation, clinical trial of an ocular drug delivery insert of the invention is being conducted to evaluate the safety of an ocular drug delivery insert containing vorolanib in the management of subjects with neovascular (wet) age-related macular degeneration (AMD). Interim 6 month results have been evaluated and reported.

At the start of the study, 17 subjects, all of whom had been previously treated for wet AMD, were enrolled. The subjects in this study had diagnosed wet AMD in the study eye for at least 4 months prior to the screening visit. To be included, subjects had to have received at least 3 previous injections with an anti-VEGF product in the study eye, such as bevacizumab (Avastin®, Genentech), ranibizumab, (Lucentis®, Genentech), or aflibercept (Eylea®, Regeneron) during the previous 6 months and a BCVA between 25 letters (20/320 Snellen equivalent) and 75 letters (20/32 Snellen equivalent).

TABLE 12
Subject Screening Characteristics (N = 17)

| | | |
|---|---|---|
| Mean age, range (years) | 77.4 | (67-94) |
| Female (n, %) | 13/17 | (76%) |
| Mean BCVA, range (ETDRS letters) | 69 letters, | (38-85) |
| Mean CST, range (microns) | 299 microns, | (204-441) |

| | |
|---|---|
| Median length of time for wet AMD | 17 months |
| diagnosis prior to enrollment | |
| Mean No. of anti-VEGF injections | 8.76 injections/year |
| per year prior to enrollment | |

For subjects with unilateral wAMD, the affected eye was designated as the study eye; for subjects with bilateral wAMD, the study eye was the more severely affected eye meeting the inclusion/exclusion criteria, i.e., the eye having the worse BCVA or if equal, the eye clinically judged to be the more severely affected eye as determined by the Investigator. If the eyes are symmetrically affected, the study eye was the right eye.

One week after screening and a standard-of-care anti-VEGF injection, the subjects received 1 injection of the study drug, an ocular drug delivery insert containing vorolanib and PVA. The study included 4 dosing cohorts: low dose, low medium dose, mid dose, and high-dose. A 25-gauge needle was used for the low dose injection and a 22-gauge needle was used for the other injections.

TABLE 13
Dosing Cohorts

| No. | | Number of | Vorolanib μg/ | No. Inserts/ |
|---|---|---|---|---|
| Subjects | Total Dose | Injections | Insert | Injection |
| 3 | Low 440 μg | 1 | 440 | 1 |
| 1 | Low Medium | 1 | 1,030 | 1 |
| | 1,030 μg | | | |
| 8 | Mid 2,060 μg | 1 | | 2 |
| 5 | High 3,090 μg | 1 | | 3 |

The duration of release of the active pharmaceutical ingredient (vorolanib) is expected to be at least 9 months. There was no reinjection of the study drug during the first 6 months of the trial.

Following injection on Study Day 0, subjects were to return on Study Days 7, 14, 28, and every 4 weeks thereafter through Month 12.

Assessments include BCVA by ETDRS, anterior/posterior segment ocular examination, IOP, fluorescein angiography (FA), color fundus photography (CFP), treatment-emergent ocular and non-ocular adverse events (TEAEs), clinical laboratory evaluations (hematology, serum chemistry, coagulation, and urinalysis), vital sign measurements (see details in attached Schedule of Study Procedures and Assessments), spectral-domain-optical coherence tomography (SD-OCT), and, at study sites where equipment is available, OCT-Angiography (OCT-A).

The primary study endpoint is to evaluate safety and determine the maximum tolerated dose for the treatment of neovascular (wet) AMD based on treatment-emergent ocular (study and fellow eye) and non-ocular adverse events (TEAEs), including clinical laboratory findings; the secondary endpoints include BCVA and CST measured by OCT. The investigators are also evaluating the number of eyes that do not require supplemental (previously referred to in our study protocols as “rescue”) treatment at various time points and the degree to which the anti-VEGF treatment burden is reduced after administration of EYP-1901.

Following the intravitreal injection of the EYP-1901 insert, an FDA-approved anti-VEGF treatment for wet AMD or off-label bevacizumab may be administered at the Investigator's discretion if at least one of the following criteria is met:
Presence of new or worsening vision-threatening hemorrhage due to wet AMD from baseline (Day 0) OR
Increase in CST of >75 μm from baseline (Day 0) OR
Loss of ≥10 ETDRS letters from baseline (Day 0) with intra-/sub-retinal fluid and/or hemorrhage judged to be the cause of BCVA loss.
   If the above criteria are not met the Investigator may still determine the need for administering a supplemental medication in the best interest of the subject's welfare.

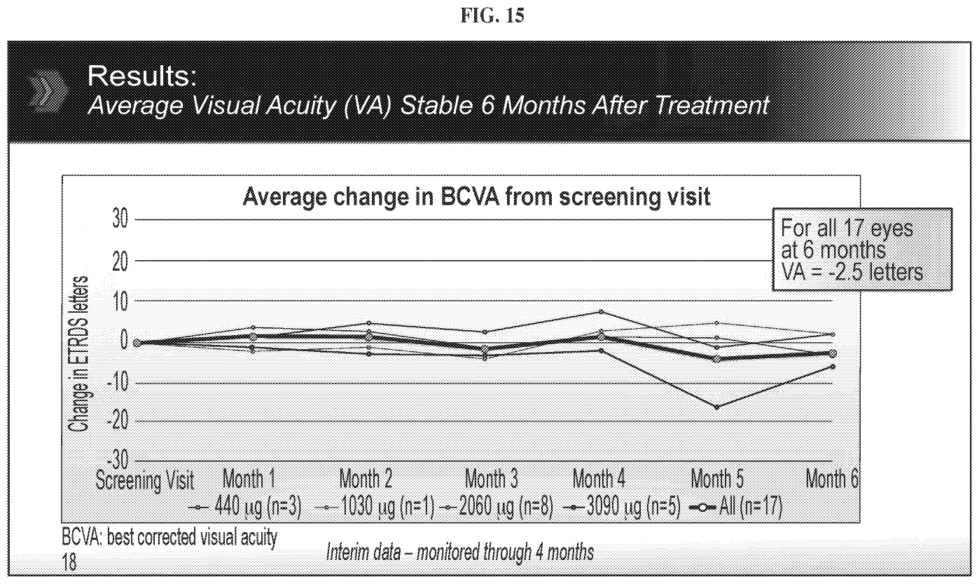

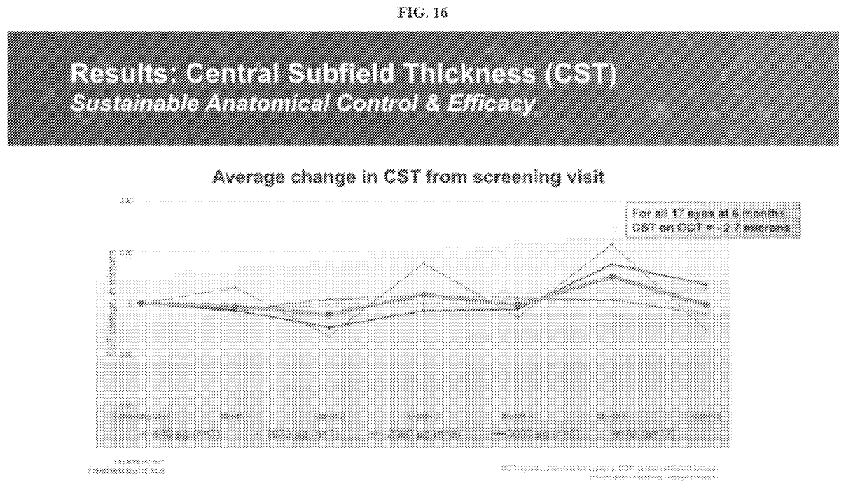

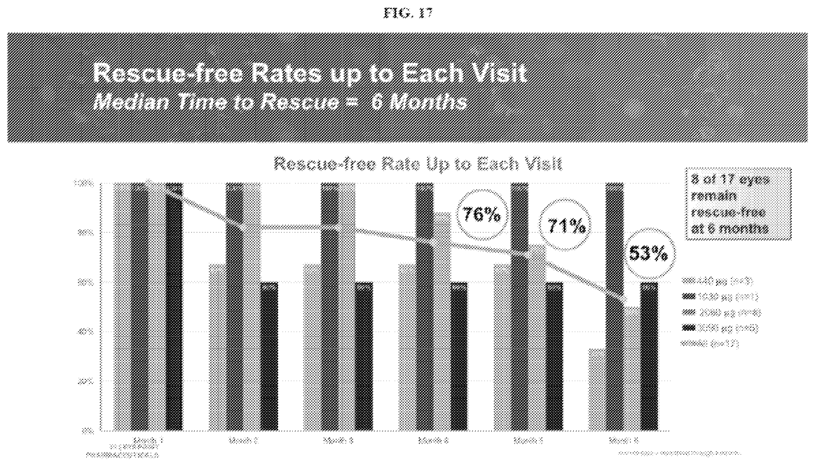

Conclusions at 6 months: The interim 6-month results from this study showed positive safety data, with no dose limiting toxicities (DLTs), no ocular serious adverse events (SAEs) reported, and no drug-related systemic SAEs reported. The majority of ocular adverse events reported were mild and to be expected. All ocular AEs were ≤grade 2, with the only grade 3 AE not being drug-related. Across all dose cohorts, 76% of subjects were supplemental-free up to 4 months, and 53% of subjects were supplemental-free up to 6 months, with a median time to supplemental treatment of 6 months across all subjects. At the six-month visits, stable best corrected visual acuity (BCVA), with an average change of −2.5 letters at 6 months, and central subfield thickness (CST), with an average change of −2.7 μm, were achieved. Three out of 17 subjects required supplemental treatment at Month 1, but these subjects would be considered suboptimal responders to standard-of-care anti-VEGF therapy as well. The overall treatment burden was reduced by 79% at 6 months across all cohorts, and 8 out of 17 subjects remain supplemental-free with one subject supplemental-free up to 9 months. The average change in BCVA from the screening visit is shown in a graph in FIG. 15. The average change in CST from the screening visit is shown in a graph in FIG. 16. The supplemental-free rate for each visit is shown in a graph in FIG. 17.

### Example 9—Evaluation of the Effect of Vorolanib on Visual Function and Structure Following Retinal Detachment in Mice

### Methods

An 18-day study was conducted in age-matched and experimentally naive C57Bl/6 mice. Prior to experiments, the mice were randomly distributed into two separate study arms, and identification numbers were assigned to individual mice. Prior to dosing initiation, baseline experiments were conducted to measure visual acuity and contrast vision by optokinetic tracking (OKT), and to measure retinal and outer nuclear layer (ONL) thickness in images obtained by optical coherence tomography (OCT). On Day 1, and continuing until Day 18, the mice received once a day oral administration of vehicle (Arm 1), or Vorolanib at 40 mg/kg (Arm 2). On Day 2, bilateral subretinal injection of sodium hyaluronate was performed to induce retinal detachment. On Day 16, visual acuity and contrast vision thresholds were measured, and on Day 17, OCT was performed to measure retinal and ONL thickness. On Day 18, terminal collections were performed at either 30 minutes, or 4 hours, post-administration, with whole blood collected and processed to plasma, left eyes collected and fixed for histology, and right eyes collected and dissected to yield cornea, lens, retinal pigment epithelium (RPE)/choroid/sclera, and retina, separately.

### Summary and Conclusion

In this mouse model, a single bilateral subretinal injection of sodium hyaluronate induces retinal detachment of the sensory retina from the retinal pigmented epithelium. This model is highly reproducible across eyes and allows for efficacy evaluation of therapeutic intervention. In this study, vorolanib was administered orally once a day for 18 days to study the effects on vision loss measured by OKT recording, and retinal degeneration as measured by OCT imaging.

Vorolanib was present in the plasma up to 4 hours following 18 consecutive days of once per day oral administration and was within detectable range in all ocular tissues tested, except lens tissue, up to 4 hours following the terminal dose. Vorolanib appears to be detected primarily in RPE/Choroid/Sclera tissue at levels that exceed plasma concentrations.

Retinal detachment induced significant loss of both visual acuity (Vehicle=36% decline; vorolanib=23% decline) and contrast vision (Vehicle=75% decline; vorolanib=41% decline) in both treatment arms. However, the drop off from baseline to the Day 16 recordings were less severe in vorolanib administered mice, and the contrast thresholds were significantly higher in the vorolanib administered mice compared to vehicle (P=0.0009).

ONL thickness did not decline in mice treated with vorolanib, suggesting a protective effect against photoreceptor degeneration. In scans acquired across both the vertical, and horizontal retinal axes, there was little difference from baseline to Day 17 in measurements of total retinal thickness within each arm, and there was no statistically significant differences in total retinal thickness across the treatment arms. However, there was a significant loss of ONL thickness in vehicle administered eyes (8% loss in vertical scans; 9% loss in horizontal scans) but not vorolanib administered eyes (1% loss in vertical scans; no loss in horizontal scans). This indicates that retinal thinning occurred primarily in photoreceptor cell bodies, and vorolanib provided specific protective efficacy for these cells.

In this study, vorolanib, administered at established therapeutic levels, provided a protective effect on loss of visual acuity and contrast vision by protecting against photoreceptor degeneration. Therefore, once a day administration for 18 consecutive days of vorolanib at 40 mg/kg provides a significant neuroprotective effect with improved visual outcomes in a mouse model of sodium Hyaluronate-induced retinal detachment compared to vehicle administration. These findings are supported by improvements in structure and function.

Thus, the results from both optokinetic tracking and OCT of ONL following retinal detachment in mouse by subretinal injection of sodium hyaluronate suggests that vorolanib has neuroprotective properties.

### Experimental Design

Baseline: Optokinetic quantification of spatial frequency threshold and contrast threshold; OCT imaging to quantify total retinal thickness and ONL thickness.

Days 1-18: Oral administration of vehicle or test agent, Quaque die (once a day).

Day 2: Bilateral subretinal injection of Hyaluronate to cause detachment.

Day 16: Optokinetic quantification of spatial frequency threshold and contrast threshold.

Day 17: OCT imaging to quantify total retinal thickness and ONL thickness.

Day 18 at 30 min±5 min following oral administration of vehicle or test agent: Terminal plasma collection; Enucleation of eyes with left eyes fixed in 10% NBF and right eyes dissected to yield cornea, lens, RPE/choroid/sclera, and retina, with tissue snap frozen in liquid N2 with tube and tissue weights recorded (n=4 mice/arm).

Day 18 at 4 hr±15 min following oral administration of vehicle or test agent: Terminal plasma collection; Enucleation of eyes with left eyes fixed in 10% NBF and right eyes dissected to yield cornea, lens, RPE/choroid/sclera, and retina, with tissue snap frozen in liquid N₂ with tube and tissue weights recorded (n=4 mice/arm).

### Methods

### Anesthesia

Ketamine and Xylazine were applied via intraperitoneal injection at 85 mg/kg and 14 mg/kg, respectively.

### Oral Gavage

Oral gavage was performed on conscious mice by trained personnel; no anesthesia was required. Delivery was achieved using standard gavage technique utilizing a 22G×1.5″ and 1.25 mm straight needle (Cadence Science) attached to a 1 mL syringe which is placed in the animal's mouth and advanced via the esophagus to the stomach where the drug is dispensed. For test agent dosing, a volume of 4 mL/kg (4 μl/g) of the formulation was dosed once daily in accordance with the Good Practice Guide (Journal of Applied Toxicology 21:15-23). Following removal of the syringe, the animal was held for another 30-60 seconds for observation of possible reflux, or signs of distress.

### Subretinal Injection of Hyaluronate

Hyaluronate (Provis Viscoelastic, Alcon Surgical, In. Cat #1314033) was shipped in a 0.85 mL syringe containing 10 mg Sodium Hyaluronate and used as is. On the first day of use, aliquots were made for future use.

Mouse eyes were dilated and the animals were anesthetized according to standard operating procedures. The mouse was placed on a regulated heating pad, and the posterior pole was visualized under magnification. A 12.7 mm 30-gauge insulin syringe was used to puncture the cornea just above the corneal limbus, avoiding any contact with the sclera and lens. The transvitreal subretinal injections were performed using a 10 μL Hamilton syringe with a 33-gauge blunt needle inserted through the corneal puncture across the vitreous, with the shaft aimed at the back of the eyecup, avoiding any trauma to the lens or iris. A total volume of 1 μL was delivered.

### Optokinetic Tracking (OKT)

OKT is performed using an OptoMotry designed for rodent use (Cerebral Mechanics Inc.). In this non-invasive assessment, mice are placed onto a platform surrounded by 4 LCD screens which reside within a light-protected box. Visual stimuli are then presented to the mouse via the LCD screens and a masked observer visualizes and scores optokinetic tracking reflexes from a digital camcorder which is mounted on the top of the box. The monitors display continuous vertical sine wave gratings rotating across the monitors at 12 degrees/s which appear to the animal as a virtual three-dimensional rotating sphere. The rotation of the virtual cylinder is constantly centered at the animal's viewing position to ensure a consistent viewing distance. Tracking movements are identified as slow, steady head movements in the direction of the rotating grating. For measurements of spatial frequency threshold, mice were tested at a range of spatial frequencies from 0.064 to 0.464 cycles/degree. The OptoMotry device employs a proprietary algorithm to accept the input from the masked observer and automatically adjusts the testing stimuli based upon whether the animal exhibited the correct or incorrect tracking reflex. All measurements of contrast threshold were performed at a spatial frequency threshold of 0.064 cycles/degree. The contrast threshold is calculated as a reciprocal of the Michelson contrast from the screen's luminance (maximum−minimum)/(maximum+minimum). The reciprocal of the contrast threshold is plotted.

### Optical Coherence Tomography

Animals were anesthetized and eyes were dilated according to standard procedures. Following sedation and dilation, animals were secured on a platform and presented to a Micron

IV OCT module (Phoenix Research Inc.) for imaging. The total retina thickness and outer nuclear layer thickness were measured from vertical and horizontal scans within each eye, and the data was plotted as the average retinal thickness across the scans.

### Plasma Collection

Following sedation by intraperitoneal (IP) administration of Ketamine/Xylazine, approximately 500 μl of terminal whole blood was collected via cardiac puncture. The blood was immediately placed in a K2EDTA tube and centrifuged at 5,000×g for 10 minutes to obtain plasma.

### Tissue Collection

Animals were sedated by IP administration of Ketamine/Xylazine and then euthanized by intracardial administration of Euthasol®.

For histopathology, following euthanasia at the designated timepoint, the superior portion of the left eyes were demarcated with a scorch mark, and the eyes were enucleated with optic nerve intact, placed in a screw cap tube, and fixed in 10% neutral buffered formalin (NBF) for 24 hours at room temperature and then transferred to 70% Ethanol and stored at 4° C. until shipment on cold packs.

For ocular PK, right eyes were enucleated at the designated timepoint, and cornea, lens, RPE/choroid/sclera, and retina, were individually dissected and snap frozen in liquid N₂.

Individual tube and tissue weights were recorded.

### Data and Statistical Analyses

Graphs were generated using GraphPad Prism (v. 9.4.0). Statistical analysis was also performed using GraphPad to generate descriptive for each data set, and to compare for statistical significance using a one-way analysis of variance for significance with Sidak's multiple comparison post-test comparing the following means: Baseline versus Day 17 within each treatment arm, Arm 1 baseline versus Arm 2 baseline, and Arm 1 Day 17 versus Arm 2 Day 17. Only changes with a p-value<0.05 are deemed statistically significant.

### Data Handling

### Data Collection and Analysis

Statistical analysis was performed on data using GraphPad Prism software (version 9.4.0).

### Results

### Optokinetic Tracking

Optokinetic tracking (OKT) was employed at baseline, and Day 16, to evaluate visual acuity by measuring the maximum spatial frequency threshold (SFT) of a grated visual stimulus at which the mice displayed a consistent and verifiable optomotor response as a measure of visual acuity, or to measure the minimum contrast in the grated stimuli at which the mice displayed an optomotor response as a function of contrast vision.

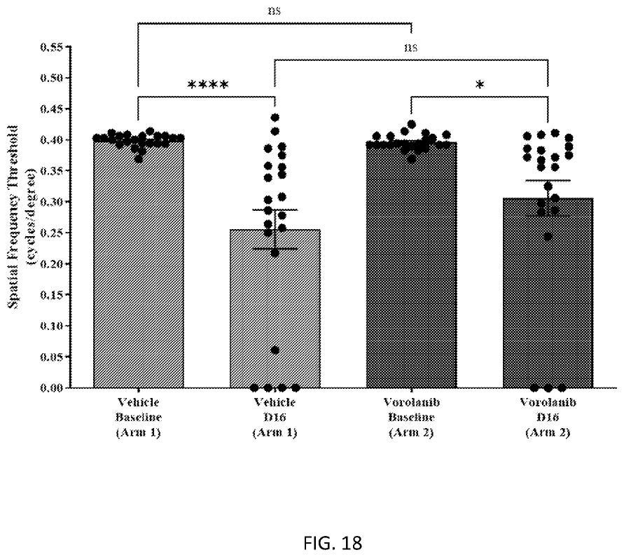

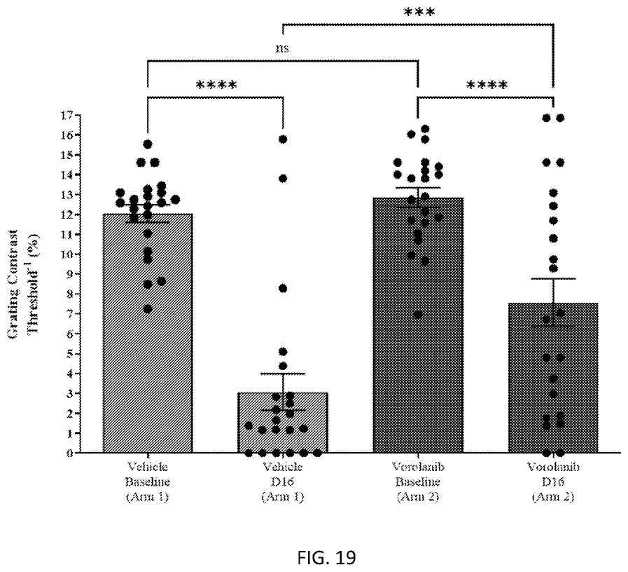

Visual acuity was significantly lower at Day 16 compared to baseline within each treatment arm (FIG. 18). The loss was greater for the vehicle administered mice than the vorolanib administered mice. Although the vorolanib mice had a higher mean SFT at Day 16 (0.306 versus 0.256 cycles/degree), the difference was not statistically significant. Similarly, the mean contrast vision threshold decreased significantly from baseline to Day 16 within each treatment arm (FIG. 19), and vorolanib administration led to a statistically significant improvement in contrast thresholds compared to vehicle (***, p=0.0009; 1-way ANOVA).

Therefore, once a day administration of vorolanib at 40 mg/kg limited the loss of visual acuity and had a significant positive effect on loss of contrast vision in this model of retinal detachment.

### OCT Imaging and Retinal Thickness Quantification

OCT imaging was used to measure retinal thickness and ONL thickness at baseline, and Day 17. Scans were acquired across either the vertical axis from the inferior to superior retina, or across the horizontal axis. For horizontal axis measurements, scans were acquired from temporal to nasal in left eyes, and nasal to temporal in right eyes.

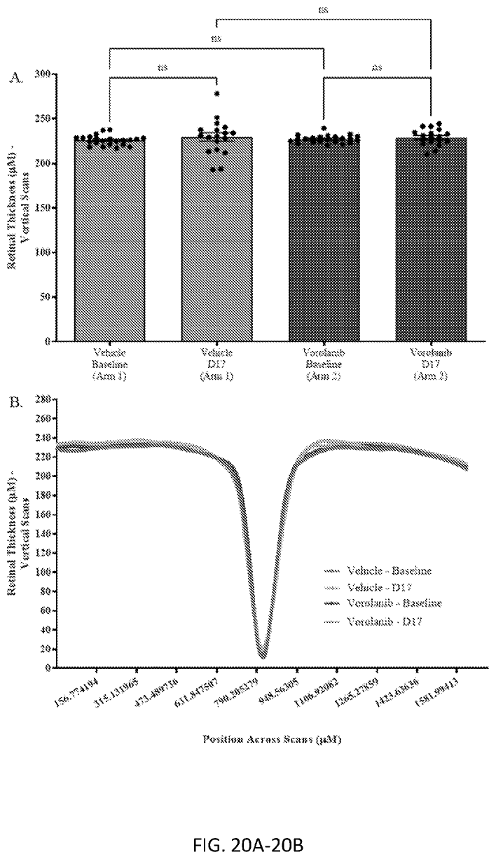

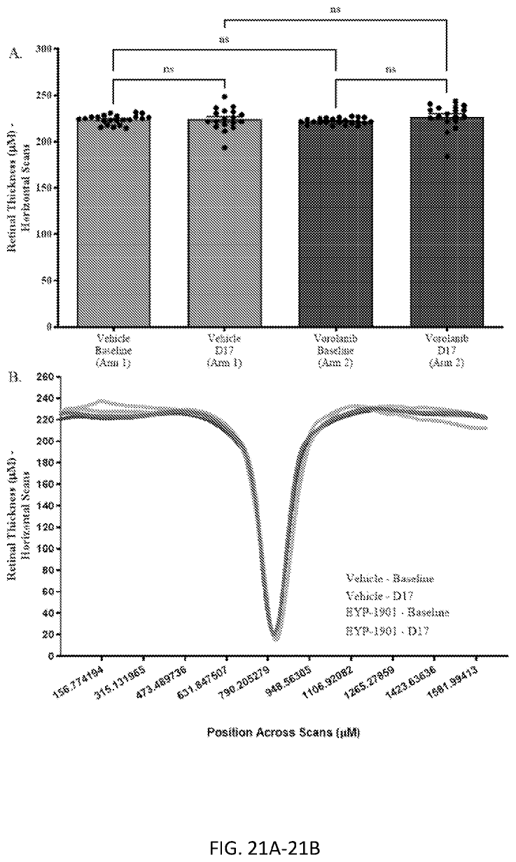

The mean retinal thickness across vertical (FIGS. 20A-20B) and horizontal (FIGS. 21A-21B) was ˜222-229 μm across both arms and timepoints. There was no statistical difference in the means compared in this study. This indicates there was minimal retinal cell death resulting from sodium hyaluronate induced retinal detachment.

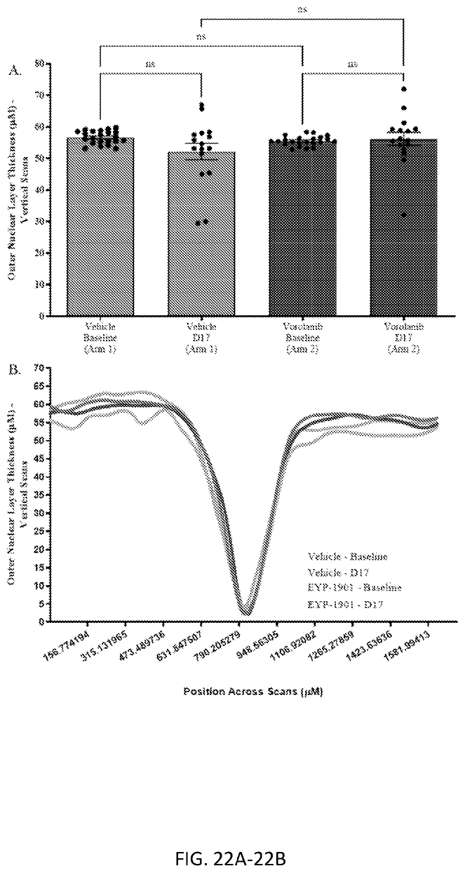

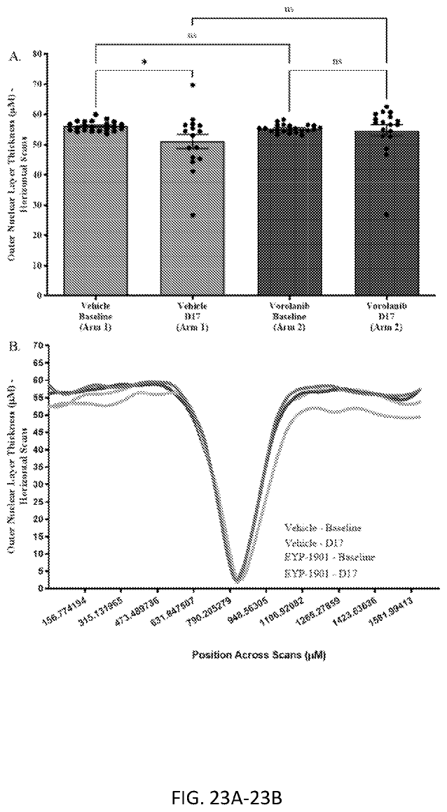

Measurement of ONL alone in vertical scans showed there was a slight, but not significant, reduction in mean thickness from baseline to Day 17 in vehicle administered eyes, but not in vorolanib administered eyes (FIGS. 22A-22B). ONL measurements in horizontal scans also showed a loss of mean thickness in vehicle eyes from baseline to Day 17, and the difference was statistically significant (FIGS. 23A-23B). There was no loss in mean ONL thickness in vorolanib administered eyes. Therefore, once a day administration of vorolanib significantly reduces the loss of ONL thickness in this mouse model of retinal detachment.

What is claimed is:

1. A method for preventing wet AMD in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days.

2. A method of preventing choroidal neovascularization in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days.

3. A method of preventing conversion of category 3 AMD to category 4 AMD in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days, wherein the eye has category 3 AMD at baseline.

4. A method of slowing the progression of AMD in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days, wherein the eye has category 2, category 3 or category 4 AMD at baseline.

5. The method of any of claims 1-4, wherein the subject has unilateral wet AMD in the subject's other eye at baseline.

6. The method of claim 1 or 2, wherein, at baseline, the eye is in category 1 or has category 2 AMD.

7. The method of any of claims 1-4, wherein the subject has category 3 AMD in the subject's other eye at baseline.

8. The method of claims 1-4, wherein the subject has category 3 AMD in both eyes at baseline.

9. The method of claims 1-4, wherein, at baseline, the eye has category 3 AMD and the subject's other eye has category 4 AMD.

10. The method of claim 1, 2 or 4, wherein, at baseline, the eye has category 2 AMD and the subject's other eye has category 4 AMD.

11. The method of claim 1 or 2, wherein, at baseline, the eye is category 1 and the subject's other eye has category 4 AMD.

12. The method of any of claims 5 and 7-11, wherein an ocular drug delivery insert comprising vorolanib, or a pharmaceutically acceptable salt thereof, that releases about 0.1 μg/day to about 40 μg/day of vorolanib for at least 90 days and is capable of at least 20% erosion within 95 days, is administered to both of the subject's eyes.

13. The method of any of claims 1-4, wherein there is no change from baseline in BCVA of the eye for 180 days starting on the day on which the insert is administered.

14. The method of any of claims 1-4, wherein, for 180 days starting on the day on which the insert is administered, the change from baseline in BCVA of the eye is a loss of ≤5 ETDRS letters.

15. The method of any of claims 1-4, wherein, for 180 days starting on the day on which the insert is administered, CST of the eye does not increase by more than 75 μm.

16. The method of claim 15, wherein CST of the eye does not increase over baseline.

17. The method of any of claims 1-4, wherein there is no detectable choroidal neovascularization in the eye for 180 days starting on the day on which the insert is administered.

18. The method of any of claims 1-4, wherein, for at least 180 days starting on the day on which the insert is administered, the eye does not progress to an AMD category higher than the eye was at baseline.

19. The method of any of claims 1-4, wherein for at least 180 days, starting on the day on which the insert is administered, the IVI questionnaire composite score for the subject does not increase significantly from baseline.

20. The method of any of claims 1-5, wherein the insert comprises a solid matrix core comprising the vorolanib, or a pharmaceutically acceptable salt thereof, and a matrix polymer.

21. The method of claim 20, wherein the matrix polymer is polyvinyl alcohol (PVA).

22. The method of claim 21, wherein the amount of matrix polymer in the insert is about 1% w/w to about 15% w/w.

23. The method of any of claim 1-5 or 22, wherein the amount of the vorolanib, or pharmaceutically acceptable salt thereof, in the insert is about 60% w/w to about 98% w/w.

24. The method of any of claim 1-5 or 22, wherein the amount of the vorolanib, or pharmaceutically acceptable salt thereof, in the insert is about 85% w/w to about 99% w/w.

25. The method of any of claim 1-4 or 21, wherein the insert is capable of at least 90% erosion within 440 days.

26. The method of any of claims 1-4, wherein the insert comprises about 200 μg to about 2000 μg of vorolanib or a pharmaceutically acceptable salt thereof.

27. The method of any of claims 1-4, wherein the insert releases about 0.5 μg/day to about 30 μg/day of vorolanib for at least 120 days.

28. The method of any of claims 1-4, wherein the insert is administered by intravitreal injection through a 20 to 27 gauge needle or cannula.

29. The method of claim 28, wherein the insert has a length of about 1 mm to about 10 mm.

30. The method of any of claims 1-29, wherein the insert further comprises a coating substantially surrounding the core.

31. The method of claim 30, wherein the insert further comprises a delivery port.

32. The method of claim 30, wherein the coating comprises PVA.

33. The method of claim 32, wherein the matrix polymer is PVA and the coating comprises a different grade of PVA than the matrix polymer.

34. The method of claim 32, wherein the coating comprises at least two coats comprising PVA, and wherein at least one of the coats comprises a different grade of PVA from at least one other coat.

35. The method of claim 32, wherein the coating comprises more than one coat comprising PVA, the matrix polymer is PVA, and the DH of the PVA in at least one coat differs from the DH of the matrix polymer PVA.

36. The method of claim 32, wherein the matrix polymer is PVA and the MW of the PVA in the coating differs from the MW of the matrix polymer.

37. The method of claim 28, wherein 1-6 inserts are injected.

38. The method of claim 37, wherein the total amount of vorolanib in all of the inserts is about 600 μg to about 6000 μg.

39. The method of claim 38, wherein the one or more ocular drug delivery inserts deliver a total average daily dose of vorolanib of about 1 μg/day to about 50 μg/day for at least 30 days.

40. The method of any of claims 1-4, wherein the insert was cured for about 200 minutes to about 1440 minutes at about 60° C. to about 120° C.

41. The method of any of claims 1-4, wherein the insert is made by dissolving PVA in an aqueous solution to form a PVA solution, mixing the PVA solution with vorolanib or a pharmaceutically acceptable salt thereof to form a matrix mixture, extruding the mixture through a dispensing tip to form an elongated shaped matrix, curing the elongated shaped matrix at a temperature of about 80° C. to about 160° C. for about 15 minutes to about 4 hours, and segmenting the elongated shaped matrix.

42. A method for preventing wet AMD in an eye in a human subject comprising administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days.

43. A method of preventing choroidal neovascularization in an eye in a human subject comprising administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days.

44. A method of preventing conversion of category 3 AMD to category 4 AMD in an eye in a human subject comprising administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days, wherein the eye has category 3 AMD at baseline.

45. A method of slowing the progression of AMD in an eye in a human subject comprising administering to the eye an ocular drug delivery insert comprising vorolanib, or a pharmaceutically acceptable salt thereof, wherein the average drug release rate over a 30 day period for the insert is about 0.1 μg/day to about 40 μg/day of vorolanib and the insert is capable of at least 20% erosion within 95 days, wherein the eye has category 2, category 3 or category 4 AMD at baseline.

46. The method of any of claims 42-45, wherein the subject has unilateral wet AMD in the subject's other eye at baseline.

47. A method of preventing the loss of visual acuity due to damage to or loss of retinal cells in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days.

48. The method of claim 47, wherein the administration of the ocular drug delivery insert reduces retinal thinning in the eye.

49. The method of claim 47, wherein the administration of the ocular drug delivery insert protects against photoreceptor degeneration in the eye.

50. A method of providing neuroprotection to an ocular tissue in an eye in a human subject comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days.

51. The method of claim 50, wherein the ocular tissue is the retina of the eye to which the drug delivery insert is administered.

52. A method of treatment or prophylaxis of an ocular disease, comprising administering to the eye an ocular drug delivery insert comprising vorolanib, or a pharmaceutically acceptable salt thereof, wherein the insert releases about 0.1 μg/day to about 60 μg/day of vorolanib for at least 90 days and the insert is capable of at least 20% erosion within 95 days,
wherein the ocular disease is characterized by damage to retinal neurons.

53. The method of claim 52, wherein the damage to retinal neurons affects photoreceptors.

54. The method of claim 52, wherein the ocular disease is geographic atrophy, glaucoma, or retinal detachment.
